(19) United States
(12) Patent Application Publication
(10) Pub. No.: US 2024/0124917 A1
(43) Pub. Date: Apr. 18, 2024

(54) COMPOSITIONS AND METHODS FOR NUCLEIC ACID DETECTION BY LATERAL FLOW ASSAYS

(71) Applicant: King Abdullah University of Science and Technology, Thuwal (SA)

(72) Inventors: Magdy Mahfouz, Thuwal (SA); Tin Marsic, Thuwal (SA)

(21) Appl. No.: 18/546,515

(22) PCT Filed: Feb. 15, 2022
(86) PCT No.: PCT/IB2022/051326

§ 371 Date: Aug. 15, 2023

Related U.S. Application Data
(60) Provisional application No. 63/149,638, filed on Feb. 15, 2021.

(51) Int. Cl. C12Q 1/6816; C12N 9/22

(52) U.S. Cl. CPC C12Q 1/6816; C12N 9/22; C07K 2319/80

(57) ABSTRACT

Rapid, sensitive, and specific point-of-care testing for pathogens is crucial for disease control. Lateral flow assays (LFAs) have been employed for nucleic acid detection, but they have limited sensitivity and specificity. A fusion of catalytically inactive Cas9 endonuclease and a relaxase for example, VirD2 are used for sensitive, specific nucleic acid detection by LFA. VirD2-dCas9 specifically binds the target nucleic acid sequence via dCas9 and covalently binds to a FAM-tagged oligonucleotide via VirD2. The biotin label and FAM tag are detected using a LFA. This system, termed Vigilant (VirD2-dCas9 guided and LFA-coupled nucleic acid test) is coupled to reverse transcription-recombinase polymerase amplification to detect pathogenic nucleic acid of interest in a sample, it exhibits an impressive limit of detection and shows no cross-reactivity, thus reducing incidents of false positives. Vigilant offers an easy-to-use, rapid, cost-effective, and robust detection platform for SARS-CoV2.

### CROSS-REFERENCE TO RELATED APPLICATION

The present application claims priority to U.S. application Ser. No. 63/149,638, filed Feb. 15, 2021, the disclosure of which is incorporated herein by reference in its entirety.

### FIELD OF THE INVENTION

The present invention is in the field of nucleic acid detection in a sample.

### BACKGROUND OF THE INVENTION

Rapid, sensitive, and specific diagnostics can detect pathogens and disease markers in humans, animals, plants, water, and the environment [1, 2], thus aiding treatment and mitigation measures. Although PCR-based and other sequence-based laboratory tests are capable of specific and sensitive detection of nucleic acids, they cannot meet the increasing demand for diagnostics, due to major drawbacks including their cost, turnaround time, the limited number of samples that can be processed, and the need for sophisticated equipment and skilled technical personnel [3]. Because of the widespread applications and the promise to improve human life, there is a pressing need for the development of point-of-care (POC) or at-home testing kits capable of detecting the presence of disease or infectious markers rapidly and with the desired sensitivity but low cost [4]. POC testing must meet the ‘ASSURED’ criteria by being Accurate, Specific, Sensitive, User-friendly, Rapid, Equipment-free, and Deliverable to end-users. These criteria have been recommended by the WHO for an effective POC test to control and manage infectious diseases, especially in epidemic or pandemic situations [5], such as COVID-19.

COVID-19 is caused by Severe acute respiratory syndrome coronavirus 2019 (SARS-CoV2), a member of the Coronaviridae family whose members pose an ongoing, major threat to public health [6]. A strategy focused on large-scale testing for timely identification and isolation of infected individuals and subsequent contact tracing has proven to be an effective method of curbing the spread of SARS-CoV-2. However, limitations in testing have hampered the implementation of this strategy. PCR-based testing is the gold standard for virus detection but suffers from major drawbacks that limit its use for effective, large-scale testing in pandemic situations. Therefore, there is a pressing need to develop POC testing modalities that can be deployed for testing on a massive scale [7]. Moreover, the availability of diagnostic platforms for broad, in-field deployment is of paramount importance in preventing the further spread of COVID-19, future pandemics [2, 4] and making accessible easy and quick detection of any human, animal or plant pathogen.

Clustered regularly interspaced short palindromic repeats (CRISPR) and CRISPR-associated (Cas) protein systems have been harnessed for gene editing and the nuclease-dead mutants (dCas9) have been used for gene regulation across diverse species [8-12]. Recently, CRISPR systems have been harnessed for diagnostics. CRISPR-Dx relies on the ability of the CRISPR system to scan the nucleic acid and find a complementary sequence to the single-guide RNA (sgRNA) of the CRISPR complex to activate the cis and trans activities of the CRISPR enzyme. Cas13, Cas12 and Cas9 enzymes have been used to develop different CRISPR- based modalities, including Sherlock, DETECTR, iSCAN, SHINE and CASLFA [1, 13-16]. These systems rely on the trans collateral activity of the CRISPR enzymes after the cis activation upon binding to the target sequence. For example, upon binding of the target DNA, Cas12a induces a nick in each of the target DNA strands, yielding a double-stranded DNA break. In addition to inducing cis-cleavage of the targeted DNA, target DNA binding induces trans-cleavage of non-target DNA. There is a pressing need to develop CRISPR systems that obviate the necessity of collateral activities, thereby expanding the power and applications of the CRISPR enzymes in diverse modalities for diagnostics.

Lateral flow assays (LFAs) have played critical roles in diagnostics but the extraordinary potential LFAs has not yet been fully exploited for widespread use in the detection of different analytes from diverse sources [23-26]. Although LFAs have been developed to detect nucleic acids, the current technologies detect the presence or absence of the target sequences irrespective of their identity or the authenticity of the sequence. For example, recombinase polymerase amplification (RPA) coupled with LFA was used for nucleic acid detection, but the primers and primer dimers could cause false positives, compromising the specificity of the assay; moreover, the addition of the expensive specificity reagent to LFAs make it unfeasible for massive testing [27]. Therefore, there is a pressing need to develop a simple, inexpensive, sensitive, and specific LFA for nucleic acid detection, which can be employed for pathogen RNA detection, including SARS-CoV2.

It is an object of the present invention to provide compositions and methods for detecting a nucleic acid from a pathogen in a sample.

It is another object of the present invention to provide compositions and methods for improved rapid and sensitive detection of viral RNA in a sample.

It is a further object of the present invention to provide compositions and methods for one-step reactions that facilitate detection of a nuclei acid of interest in a sample.

### SUMMARY OF THE INVENTION

A nucleic acid detection platform is disclosed, which employs chimeric fusions between a CRISPR enzyme and a relaxase to provide the dual functions of specific binding to the target sequence and the binding to a ssDNA probe resulting in a molecular complex for LFA detection.

The CRISPR enzyme can be a Cas9 variant, Cas12 or Cas14 variants. In some embodiments, the detection system uses a PAM-independent Cas9 variants capable of the recognition of any DNA sequence in a PAM-independent manner. This bypasses the need to find a PAM sequence for the binding site and expand the utility of this platform to any nucleic acid sequence. Relaxases with different recognition sequences can be employed for binding to a ssDNA probe. In some embodiments, the relaxase is the. VirD2 relaxase.

The disclosed compositions and methods employ the dual functions of the CRISPR/Cas9 enzyme for DNA scanning and recognition and the relaxase, for example, VirD2 relaxase for covalent binding to a single-stranded DNA (ssDNA) probe, coupled with LFA for virus detection. A chimeric fusion between dCas9 and VirD2 coupled with ssDNA reporter as a detection complex is disclosed. The Vigilant system provides a sensitive, specific, and low-cost modality for COVID-19 detection, and nucleic acid detection in general, which can be employed as a POC test.

Also disclosed is method of detecting presence of a nucleic acid in a sample by combining the sample and plurality of primers specific to the nucleic acid with a disclosed composition under conditions sufficient for amplification of the nucleic acid, and detecting the nucleic acid amplification product, thereby detecting presence of the nucleic acid in the sample. In some embodiments, the nucleic acid is derived from a coronavirus, preferably a severe acute respiratory syndrome-related coronavirus, such as SARS-CoV-2.

Methods of diagnosis are also provided, such as methods of diagnosing infection with a pathogen and methods of detecting the presence of a pathogen of interest. In preferred embodiments, the pathogen is a virus (e.g., SARS-CoV-2). In particular, disclosed is a method of diagnosing a subject as infected with a virus by detecting the presence of a viral nucleic acid in a sample from the subject by performing any of the aforementioned methods. Typically, detecting an amplification product indicates that the subject is infected with the virus. The subject may or may not exhibit one or more symptoms of a disease, disorder, or condition associated with the virus. In some embodiments, the method further includes treating the subject, where the subject was diagnosed as infected with the virus. Preferably, the subject is human.

In any of the foregoing methods, the sample can be an RNA sample derived from mucus, sputum (processed or unprocessed), saliva, bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood. The sample can be one that is isolated from a subject that may have been exposed to or is suspected of having SARS-CoV-2. In some embodiments, the sample is processed to expose or isolate nucleic acids from sample before it is subjected to the detection, one-step RT-PCR, or other method.

Thus, in some embodiments, the methods include collecting one or more samples from a subject and/or extracting and purifying RNA from the samples. Typically, the purified RNAs are converted to DNA by a reverse transcription reaction using reverse transcriptase (Reverse transcription). At this stage, if the subject was infected with viral RNA for example, the complementary DNA (cDNA) fragments derived from the RNA viruses are generated. Then, the virus-originated DNA fragments are sufficiently amplified by the qPCR reaction to a detectable level (e.g., by a fluorescent signals). The one-step RT-qPCR platform can simultaneously achieve both the RT and qPCR reactions in a single tube.

Additional advantages of the disclosed methods and compositions will be set forth in part in the description which follows, and in part will be understood from the description, or can be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE FIGURES

The accompanying drawings, which are incorporated in and constitute a part of this specification, illustrate several embodiments of the disclosed methods and compositions and together with the description, serve to explain the principles of the disclosed methods and compositions.

FIG. 1A is a schematic showing cleavage of ssDNA by VirD2. VirD2 alone is capable of cleaving ssDNA containing the specific RB recognition sequence. Following the cleavage of the oligonucleotide, VirD2 remains covalently bound to Tyr29 moiety, leaving three nucleotides of the original sequence. This property can be exploited to attach labels at the 3′ end of oligonucleotide sequence bound to VirD2. FIG. 1B-C are blots showing Covalent binding of VirD2 and ssDNA probe. Biotin labeled probe harboring the T-DNA right border sequence was incubated with fusion proteins in the presence of Mg^{2+.} VirD2-Cas9, Cas9-VirD2, VirD2-dCas9, dCas9-VirD2 bound to biotin labeled probe were detected by western blot. Biotin labeled probe, unlabeled probe, and dCas9-dVirD2 (no binding to RB sequence containing probe) were used as experimental control. Arrow head indicated the immuno-detection of the biotin labeled probe bounded to fusion proteins. FIG. 1D and 1E show covalent binding of VirD2 and ssDNA. ssDNA strand (64 bp), harboring the T-DNA right border sequence was incubated with fusion proteins in the presence of Mg2+. VirD2-Cas9, Cas9-VirD2, VirD2-dCas9, dCas9-VirD2 bound to biotin labeled probe were detected by Coomassie staining. RB containing probe did not bind dCas9-dVirD2. Red arrowhead indicated the ssDNA-fusion protein complex. FIG. 1F Confirmation of VirD2-Cas9 and Cas9-VirD2 nuclease activity. N-gene target DNA was incubated with fusion proteins in the presence sgRNA and Mg2+. Samples without sgRNA were used as controls. Arrow heads, indicate the respective DNA fragments. FIGS. 1G-I are schematics of the Vigilant platform. FIG. 1G. Reporter complex (FAM-probe—VirD2-dCas9—sgRNA) assembly. FIG. 1H. SARS-CoV-2 RNA is reverse transcribed and amplified by RT-RPA (reverse transcription recombinase polymerase amplification) using biotin-labeled primers. The biotin-labeled amplicon is then mixed with the preassembled reporter complex. Upon target recognition via the sgRNA, Cas9 remains stably bound to the DNA, yielding a complex that is labeled with both FAM (probe) and biotin (target). FIG. 1I is a schematic showing a Lateral flow assay. The streptavidin-coated line (T) captures biotin-labelled amplicons. In a sample containing SARS-CoV-2, the reporter complex bound to the target DNA will accumulate gold (Au) nanoparticle-labeled anti-FAM, resulting in the visual detection at the test line. The control line (C) is impregnated with anti-anti FAM antibodies that also accumulate Au nanoparticle-labeled anti-FAM and thus serve as a positive control. Detection of the positive samples is achieved by running the lateral flow after isothermal amplification and incubation with the reporter complex. The positive reaction is indicated by the presence of the lower (test) band while the upper band represents the control band. FIG. 1J is shows exemplary components of the test line and the control line. FIG. 1K is a schematic showing the workflow from sample collection to results.

FIG. 2A-2C. Vigilant platform for nucleic acid detection. FIG. 2A. Proof-of-concept of the Vigilant platform. SARS-CoV-2 N gene PCR amplicons were used as detection targets. PCR product (5 μL) was challenged with the preassembled reporter complex. Reactions with nonspecific N-gene target and nonspecific sgRNA, no target control, no sgRNA and no reporter complex were used as controls. FIG. 2B. Selection of the optimal fusion and probe. PCR product (5 μL) was challenged with the preassembled reporter complexes made with VirD2-Cas9 or Cas9-VirD2. Two probes, having 5 T nucleotides (probe 1) and 10 T nucleotides (probe 2) were used in the assembly of the reporter complex. Reactions with no sgRNA, no target control, or unlabeled target were used as controls. FIG. 2C. Buffer composition optimization. Four buffers, having different compositions (see Methods) were used. Buffer 4 with BSA added to the running buffer was selected based on the enhanced signal detection and lower nonspecific background. N-gene target 1; specific target, N-gene target 2; nonspecific N-gene target, N-gene-sgRNA1; specific sgRNA, VirD2-Cas9; fusion of wild-type VirD2 to wild-type Cas9. FIG. 2D. Selection of the optimal RT-RPA primer set. N-gene target DNA was amplified by RT-RPA kit using manufacturer's instructions. Black arrow heads indicate the expected amplicons. Red arrowhead, indicates the selected primer set. Primer set was selected based on the absence of non-specific amplicons and primer dimers. FIG. 2E. Selection of the optimal reaction buffer. 1, specific target sample. 2, non-specific target sample. 3, non-specific sgRNA. FIG. 2F. Selection of the optimal reporter complex concentration. Protien:sgRNA:FAM-probe were mixed in 1:1:1 ratio in 50, 100, 250, 500 nM concentration in a 25 μL reaction volume. FIG. 2G. Selection of the optimal RT-RPA product volume for detection. Different volumes (0, 1, 2, 3, 4, 5 μL) of the RT-RPA product were added to the preassembled reporter complex. No template and no sgRNA were used as negative controls. FIG. 2H. Selection of the optimal duration for detection. RT-RPA product was incubated with preassembled reporter complex for different time periods. Incubation at 37° C. for 10 minutes followed by 1 minute at 60° C. results in robust, rapid and consistent appearance of the bands at the test line. FIG. 21. Evaluation of the Vigilante at 42° C. All steps, the RT-RPA reaction, reporter complex assembly and detection reaction were carried out at 42° C. N-gene specific target and specific sgRNA were used as positive and no template samples was used as negative control.

FIG. 3A-C. Optimization of the Vigilant platform for detection of synthetic nucleic acid amplicons. FIG. 3A. Proof-of-concept of the Vigilant platform. SARS-CoV-2 (synthetic) N-gene PCR amplicons were used as detection targets. PCR product (5 μL) was challenged with the preassembled reporter complex. Nonspecific N-gene target and nonspecific sgRNA, no target control, no sgRNA, and no reporter complex were used as controls. FIG. 3B. Selection of the optimal fusion and probe. PCR product (5 μL) was challenged with the preassembled reporter complexes made with VirD2-Cas9 or Cas9-VirD2. Two probes, having 5 T nucleotides (probe 1) or 10 T nucleotides (probe 2) were used in the assembly of reporter complex. Reactions with no sgRNA, no target control, or unlabeled target were used as controls. FIG. 3C. The Vigilant platform specifically detected SARS-CoV-2. RT-RPA was performed using SARS-CoV, SARSCoV-2, MERS-CoV, TMV, and PVY as templates. RT-RPA product (5 μL) was challenged with the preassembled reporter complex. The Vigilant platform specifically detected only SARS-CoV-2. FIG. 3D and 3E. Comparison of VirD2-Cas9 and VirD2-dCas9. SARS-CoV-2 RT-RPA amplified N-gene product was detected with the Vigilante platform. N-gene target 1, N-gene specific target; N-gene target 2, N-gene non-specific target; N-gene sgRNA 1, specific sgRNA; N-gene sgRNA 2, non-specific sgRNA FIG. 3F. Reporter complex stability. Protein:sgRNA:FAM-probe were mixed in 1:1:1 ratio in 250, nM concentration and incubated at 37° C. for 60 minutes. The reporter complex was stored at different temperatures for different periods of time. N-gene specific target and specific sgRNA were used as positive and no template samples was used as negative control.

FIG. 4A-C. RT-RPA coupled with Vigilant for SARS-Cov2 detection. FIG. 4A. RT-RPA product detection with the Vigilant platform. SARS-CoV-2 (synthetic) N-gene RT-RPA amplified product used as detection targets. RT-RPA product (5 μL) was challenged with the preassembled reporter complex. Nonspecific N-gene target and two nonspecific sgRNAs, no target control, no sgRNA, and no reporter complex were used as controls. FIG. 4B. The Vigilant platform is compatible with VirD2-dCas9. Fusion proteins, VirD2-dCas9, dCas9-VirD2 and dCas9-dVirD2 were purified and evaluated for the Vigilant platform. RT-RPA product (5 μL) was challenged with the preassembled reporter complexes made with VirD2-dCas9 or dCas9-VirD2 or dCas9-dVirD2. VirD2-dCas9 demonstrated superior performance compared to the other two fusion proteins. FIG. 4C-4D show LoD experiment conducted in FIG. 4A FIG. 4E shows copy number determination by RT-qPCR for Ct value relevance in clinical samples. Synthetic SARS-CoV-2 RNA template was used to determine the LOD by RT-qPCR using One-step RT-qPCR kit (Invitrogen). Ct value was determined using two independent sets primer sets, N1 and N2. Blank sample (no template) was used as negative control.

FIG. 5A-C. Validation of the Vigilant platform in COVID-19 clinical samples. FIG. 5A. Detection of SARS-CoV-2 in clinical samples. RT-RPA was performed for detection of SARS-CoV-2. SARS-CoV-2 RNA was isolated with the Trizol method. Sample with viral load (Ct value, 16-38) were detected with the Vigilant platform. The N-gene RT-RPA amplified product (5 μL) was subjected to the preassembled reporter complex. Samples with Ct value >38 were considered as negative. FIG. 5B. iSCAN for the clinical samples. RT-LAMP based iSCAN was performed to confirm the Vigilant results. FIG. 5C. Table representing experimental agreement of Vigilant and RT-qPCR. FIG. 5D shows Schematic of POC utility of the Vigilant platform. After sample is collected from saliva or nasopharyngeal swab and transported in VTM, RNA is extracted and used as input for detection using Vigilant. In the first step RT-RPA with biotin-labeled primer is performed to amplify the viral genome. The resulting amplicons are then detected using the preassembled reported complex and visualized using LFA strips. FIG. 5E-F shows additional evaluated clinical samples and their original scan.

### DETAILED DESCRIPTION OF THE INVENTION

Compositions and detection principles and methods are disclosed for in-field large-scale screening and improve POC nucleic acid diagnostics at a massive scale in low resource settings, and to serve in controlling, managing, and mitigating the effects of COVID-19 or future pandemics.

POC testing is critical to mitigate, manage, and control the COVID-19 pandemic specifically, and the mass spread of any pathogen, in general. POC testing with ASSURED (Affordable, Sensitive, Specific, User-friendly, Rapid and robust, Equipment-free and Deliverable to end-users) criteria may help in reopening of the economy and facilitate trade, travel, and other human activities as well as prevent mass spreading of pathogens in/between humans, animals and plants.

The disclosed compositions and methods are low cost, sensitive, specific, and field deployable. In particular embodiments, a LFA that uses a fusion of VirD2 and dCas9 for nucleic acid detection, referred to herein as Vigilant has been developed. Vigilant can be used as a simple, affordable, and robust virus detection platform by coupling the system with RT-RPA (reverse transcriptase recombinase polymerase amplification) reactions. The example demonstrates the performance of Vigilant usingSARS-CoV2 detection in clinical samples as an example, however, Vigilant can be used to detect any user-defined nucleic acid sequence.

Current methods involving direct coupling of the LFA to amplified nucleic acid exhibit high rates of false positives due to the formation of primer dimers and non-specific binding of the probe reporter; frequent false positives also can arise from cross-contamination and non-specific amplification. Some methods attempt to address these issues. For example, the LFA-based detection platform called CASLFA exploited the ability of Cas9 to remain tightly bound to its target for hours after cleavage; FELUDA, combines isothermal amplification of target nucleic acid with the DNA recognition and unwinding activity of Cas9. The resulting product can be detected on a specially designed lateral flow strip by using specific hybridization probes immobilized on gold nanoparticles. However, these platforms require complicated reagents that are uncommon in most laboratories, difficult, laborious, and expensive to prepare, as well as custom-made reporters or lateral flow strips.

### I. DEFINITIONS

“Unit” or “U” when used in context of an enzyme refers to an amount of enzyme required to convert a given amount of reactant to a product using a defined time and temperature.

As used herein, the term “detect”, “detecting”, “determine” or “determining” generally refers to obtaining information. Detecting or determining can utilize any of a variety of techniques available to those skilled in the art, including for example specific techniques explicitly referred to herein. Detecting or determining may involve manipulation of a physical sample, consideration and/or manipulation of data or information, for example utilizing a computer or other processing unit adapted to perform a relevant analysis, and/or receiving relevant information and/or materials from a source. Detecting or determining may also mean comparing an obtained value to a known value, such as a known test value, a known control value, or a threshold value. Detecting or determining may also mean forming a conclusion based on the difference between the obtained value and the known value.

As used herein, the term “sample” refers to body fluids, body smears, cells, tissues, organs or portion thereof isolated from a subject. A sample may be a single cell or a plurality of cells. A sample may be a specimen obtained by biopsy (e.g., surgical biopsy). A sample may be one or more of cells, tissue, serum, plasma, urine, spittle, sputum, stool, swab, blood, other bodily fluid, or exudate. In some embodiments, a sample includes nucleic acids, for example, viral DNA, viral RNA, or cDNA reverse transcribed from viral RNA. The sample can be used directly (e.g., fresh or frozen), or can be manipulated prior to use, for example, by heat-treatment, purification of nucleic acids, fixation (e.g., using formalin), and/or embedding in wax (such as FFPE tissue samples).

The terms “contact”, “contacting” or “combining” describe placement in physical association for example, in solid and/or liquid form. For example, contacting or combining can occur in vitro with one or more primers and/or probes and a biological sample (such as a sample including nucleic acids) in solution.

### II. COMPOSITIONS

Vigilant requires a combination of protein activities selected to provide a nucleic acid platform that shows improved specificity and reduced assay complexity. The Vigilant system requires a fusion of catalytically inactive a chimeric fusion protein of a CRISPR cas enzyme, a relaxase, a nucleic acid sequence specifically recognized by the relaxase, that is labelled (ssDNA probe) and a Lateral How assay strip.

### A. Reporter Complex Components

A reporter complex is assembled for used in the disclosed methods and it includes a fusion protein made of Cas-relaxase, an sgRNA specific for the gene of interest, and a nucleic acid (labelled) (referred to herein as the reporter) to which the relaxase specifically binds. The components for forming the reporter complex, such as the Cas-relaxase fusion protein and the nucleic acid (labelled) (referred to herein as the reporter) to which the relaxase specifically binds, can be provided in a kit, with instructions on how to assemble the reporter complex components.

i. Fusion Proteins

Chimeric fusions of the Cas protein and relaxase. The orientation of the fusion protein can be Cas-relaxase or relaxase-Cas, and is preferably, relaxase-Cas. Thus, CRISPR cas enzyme-relaxase fusion protein represented by the general formula:

CAS-relaxase Formula I

or

relaxase-Cas Formula II,
wherein CAS is the Crispr-case enzyme. In one preferred embodiment, the orientation of the fusion protein is as shown in formula II.

Using dCas9 and VirD2 as examples, the fusion protein orientation is preferably VirD2-Cas9, as opposed to Cas9-Vird2.

### Relaxases

A relaxase is a single-strand DNA transesterase enzyme produced by some prokaryotes and viruses. In one embodiment the relaxase is a bacterial relaxase. Bacterial relaxases are enzymes that catalyze a site- and DNA-strand-specific cleavage and help to pilot the transfer of DNA across bacterial cells or other species. Agrobacterium tumefaciens, a natural genetic engineer, transfers a piece of its Ti plasmid, transferred DNA (T-DNA), into the plant genome. Upon Agrobacterium infection, a relaxosome complex of VirD1 and VirD2 binds to the Ti plasmid, and VirD2 cleaves the bottom strand of the Ti plasmid in the left and right borders. Interestingly, VirD2 remains covalently bound to the 5′ end of the single-stranded T-DNA through tyrosine 29 [17, 18]. Several relaxases have been biochemically characterized and their target sequence identified. Relaxase enzymes have been isolated from a variety of plasmid systems and shown to be activein vitroin the absence of the other components of the relaxosome. (Reviewed in Byrd, et al., Molecular Microbioll. 25(6):1011-1022 (1997); Garcilla-Barcia, et al., FEMS Microbiol Rev 33 (2009) 657-687). Examples include, but are not limited to TrwC relaxase from plasmid R388 (Escherichia coli plasmid R388 trwB gene and trwC gene; GenBank: X63150.3); RepA relaxase (Bifidobacterium adolescentis; EMBL :OSG84692.1 (ENA); UNIPROT gene: B0487_2179) , pCU1 relaxase, R1162 MobA relaxase. etc. Nash, et al., Nucleic acids Rese, 38:5929-5943 (2010). Nucleic acids to which TrwC binds are disclosed for example in Gonzalez-Perez., Embo J, 26:3847-3857 (2007) and include TCGTATTGTCTATAGCCCAGATTTAAGGA (SEQ ID NO:24), TGCGTATTGTCTATAG (SEQ ID NO:25). Sequences to which the pCU1 relaxase binds include TGTGATAGCGTGATTTATCGCGCTGCGTTAGGTGTATAGCAG (SEQ ID NO:26) (Nash, et al., Nucleic acids Rese, 38:5929-5943 (2010)).

### CRISPR Enzyme Component

In some embodiments, the CRISPR enzyme is mutated with respect to a corresponding wild-type enzyme such that the mutated CRISPR enzyme lacks the ability to cleave one or both strands of a target polynucleotide containing a target sequence. Cas proteins devoid of nucleolytic activity (dead Cas proteins; dCas) are known. Inactivation through mutation of both nuclease domains generates a catalytically dead Cas. The CRISPR enzyme can be Cas9, dCas9 (dead Cas9), dCas12, Cas12a, Cas12b, or Cpf1. Cas9 is a dual-RNA-guided nuclease that utilizes both CRISPR RNA (crRNA) and tracrRNA5 and contains both HNH and RuvC nuclease domains. Catalytically inactive, or “dead,” Cas9 (dCAS9) is mutated version of the Cas9 protein cannot cut, but still binds tightly to a particular DNA sequence specified by the guide RNA. An example is Streptococcus pyogenes Cas9 (SpCas9) can also be used in its deactivated form (SpdCas9) i.e. deactivated SpdCas9. In one embodiment, the Cas9 is a PAM independent variant capable of the recognition of any DNA sequence in a PAM-independent manner CRISPR-Cas9 mechanisms recognize DNA targets that are complementary to a short CRISPR RNA (crRNA) sequence. The part of the crRNA sequence that is complementary to the target sequence is known as a spacer. In order for Cas9 to function, it also requires a specific protospacer adjacent motif (PAM) which is usually a 2-6 basepair DNA sequence immediately following the DNA sequence targeted by the Cas9 nuclease. This embodiment uses Cas9 variants that are PAM independent.

Since the initial reports of programmed DNA cleavage by Cas9 nuclease from Streptococcus pyogenes (SpCas9) in vitrol and in mammalian cells many more Cas9 variants have been discovered and tested for genome editing, including orthologs from Staphylococcus aureus, Streptococcus thermophilus, Neisseria Meningitidis, Campylobacter jejuni and many other organisms. Cas variants are reviewed in Anzalone, et al., Nature Biotechnology 38:824-844 (2020), and exemplary variants that can be used with Vigilant include, but are not limited to SpCas9-EQR, SpCas9-VQR and SpCas9-VRER, mutants of SpCas9 that recognize NGAG, NGA and NGCG PAM sequences, respectively, or dead variants thereof; xCas9-3.7, which displays higher activity on non-NGG PAM sequences (especially NGT and NGA PAMs) than that of wild-type SpCas9. Recently, a single-point-mutation variant of SpCas9 (R691A), named HiFiCas9, has been shown to display even lower off-target editing when used with RNP delivery platforms (Vakulskas, et al., Nat. Med. 24, 1216-1224 (2018).

Cas12a is a single-RNA-guided nuclease which utilizes crRNA and contains a single RuvC domain. Cas12b is a dual-RNA-guided nuclease containing a single RuvC domain and utilizing both crRNA and tracrRNAs. See, e.g., Strecker, et al., Nature Communications, 10 (1): 212. doi: 10.1038/s41467-018-08224-4. (2019). Alteration or mutation of the RuvC domain leads to the formation of a DNAse dead Cas12a (dCas12a) which retains the crRNA processing activity of Cas12a but fails to cleave the DNA (Zetsche et al., 2015; Cell 163, 759-771. doi: 10.1016/j.cell.2015.09.038).

Functional Cpf1 does not need the tracrRNA, only crRNA is needed, utilizes a T-rich PAM and cleaves DNA via a staggered DNA DSB (4-5 nt long sticky ends, instead of blunt ends produced by Cas9). This benefits genome editing because Cpf1 is not only smaller than Cas9, but also has a smaller sgRNA molecule (proximately half as many nucleotides as Cas9), and sticky ends enhance the efficiency of genetic insertions and specificity during NHEJ or HDR.

Exemplary fusion mutant proteins include:
3_SpdCas9-dVirD2-Mutated, encoded by SEQ ID NO: 16; 5_VirD2-SpdCas9-Mutated, encoded by (SEQ ID NO:17); and 7_SpdCas9-VirD2-Mutated, encoded by SEQ ID NO:18.

Fusion proteins including the Cas protein and Relaxase can be made using methods known in the art, and include chemical synthesis, and an recombinant production in a host cell.

To recombinantly produce a fusion protein, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the fusion protein. Regulatory sequences (also referred to herein as expression control sequences) typically do not encode a gene product, but instead affect the expression of the nucleic acid sequences to which they are operably linked. The nucleotide sequences encoding the fusion protein are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The vector is preferably an expression vector in which the DNA sequence encoding the fusion protein is operably linked to additional segments required for transcription of the DNA. In general, the expression vector is derived from plasmid or viral DNA or may contain elements of both. The term, “operably linked” indicates that the segments are arranged so that they function in concert for their intended purposes, e.g. transcription initiates in a promoter and proceeds through the DNA sequence coding for the fusion protein. Expression vectors for use in expressing the fusion protein will comprise a promoter capable of directing the transcription of a cloned gene or cDNA. The promoter may he any DNA sequence, which shows transcriptional activity in the host cell of choice and may be derived from genes encoding proteins either homologous or heterologous to the host cell. Expression vectors for use in expressing the fusion protein will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell. Examples of suitable promoters for directing the transcription of the DNA in mammalian cells are the SV40 promoter (Subramani et al., Mol. Cell. Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express fusion proteins. Viral based expression systems are well known in the art and include, but are not limited to, baculoviral, SV40, retroviral, or vaccinia based viral vectors.

The expressed tagged or fusion proteins produced by the cells may be recovered from the culture medium by conventional procedures including separating the host cells from the medium by centrifugation or filtration, releasing the fusion protein by mechanical cell disruption, such as ultrasonication or pressure, precipitating the protein aqueous components of the supernatant or filtrate by means of a salt, e.g. ammonium sulphate. After sonication a suitable concentration of NaCl can be added to further decrease the ability of host cell contaminants to hind to the cation exchange matrix. After cation-exchange chromatography the fusion protein may be eluted in a salt gradient and eluate fractions containing the fusion protein are collected. In some preferred forms, fusion protein is captured from lysate through its His tag. So IMAC (immobilized metal affinity chromatography) was used and then, after concentration of protein-containing fractions, they are subjected to size exclusion chromatography (SEC) for final purification. In particularly preferred embodiments for nanobody purification, the nanobody is purified from the periplasmic space, where the host cell is bacteria, for example, E. coli. This would include (1) centrifugation, (2) osmotic shock to release the protein from the cell wall compartment, (3) IMAC (Immobilized Metal Ion Affinity Chromatography), (4) SEC (Size Exclusion Chromatography).

ii. Reporter Nucleic Acid

The reporter nucleic acid is a nucleic acid to which the relaxase enzyme specifically binds, including a label at its 3′ end. In one embodiment where the relaxase is VirD2, the reporter nucleic acid includes the 25 bp VirD2 recognition sequence GTTTACCCGCCAATATATCCTGTCA (SEQ ID NO:19) and a nucleotide bridge, for example, 5T (TTTTT) (SEQ ID NO: 20) to 15T (TTTTTTTTTTTTTTT; SEQ ID NO:21) spacer, preferably a 5T to 10T (TTTTTTTTTT; SEQ ID NO:22).

As exemplified for VirD2, the ssDNA probe consists of a 25-bp T-DNA right border VirD2 recognition site at the 5′ end and a 5-bp dT stretch with FAM at its 3′ end. VirD2 recognizes the specific 25-bp motif in the ssDNA probe, cleaves it, and remains covalently bound to the 5′ end of the ssDNA probe. dCas9 uses the specific sgRNA (5′ccaga.agctggacttccc taGrITIT AGAGCTAGAAATA GC AAGTTAAAATA AGGCTAGTCCGTTATCAACTTGAAAAAGTGGCACCGAGTCGGTGC -3′ (SEQ ID NO:23)) to target the SARS-CoV-2 N gene.

Exemplary primers are provided in Table 1.

There are two main: types of lateral flow nucleic acid tests, referred to as Nucleic Acid Lateral Flow (NALF) and Nucleic Acid Lateral Flow ImmunoAssay (NALFIA); NALF directly detects DNA/RNA exploiting capture and labelled reporter oligonucleotide probes, whereas NALFIA detects hapten-labelled DNA/RNA using capture and labelled reporter antibodies or streptavtdin.

Suitable labels that can be included in the reporter complex include but are not limited to fluorescein dye and its derivatives. In one embodiment, the label is FAM (Fluorescein amidite). Fluorescein amidites are synthetic equivalents of fluorescein dye. Other examples include the use of fluorescein isothiocyanate (FITC) labelled nucleic acids, which can be detected using immobilized anti-FITC antibodies (which are known) and coupled to colored latex mieroparticles. Other labels used to label nucleic acids are known and include, but are not limited to 7-Diethylamino-3-[4-(iodoacetamido)phenyl]-4-methylcoumarin (DCIA), lucifer yellow iodoacetamide; 5-(iodoacetamido)fluorescein (5-IAF); Oregon Green iodoacetamide; Texas Red bromoacetamide, NIR-664 iodoacetamide, etc. The lateral device is designed to include a binding partner such as an antibody that binds to the specific label used, at the control

iii. sgRNA

The CRISPR-Cas system relies on two main components: a guide RNA (gRNA) and CRISPR-associated (Cas) nuclease, which together form a ribinucleorotein (RNP) complex. The gRNA is a specific RNA sequence that recognizes the target DNA region of interest and directs the Cas nuclease there for editing. The gRNA is made up of two parts: crispr RNA (crRNA), a 17-20 nucleotide sequence complementary to the target DNA, and a tracr RNA (transactivating crRNA), which serves as a binding scaffold for the Cas nuclease. crRNAs and tracrRNAs exist as two separate RNA molecules in nature. By contrast, sgRNA (single guide RNA) is single RNA molecule that contains both a custom-designed short crRNA sequence fused to the scaffold tracrRNA sequence. sgRNA can be synthetically generated or made in vitro or in vivo from a DNA template. The CRISPR-associated protein is a non-specific endonuclease. It is directed to the specific DNA locus by a sgRNA, where it makes a double-strand break.

sgRNA design implements finding target sites in the genome by scanning PAM sequence (like 5′-NGG-3′ for SpCas9). Once the target gene and Cas nuclease have been selected, known methods can be used to design the specific guide RNA sequence. Tools for CRISPR/Cas9 sgRNA design are known in the art and are reviewed in Cui, et at., Interdisciplinary Sciences: Computational Life Sciences. 10:455-465 (2018). Methods for making sgRNA are known and include synthetically generating the sgRNA or by making the guide in vivo or in vitro, starting from a DNA template. One method involves expressing the guide RNA sequence in cells from a transfected plasmid. In this method, the sgRNA sequence is cloned into a plasmid vector, which is then introduced into cells. The cells use their normal RNA polymerase enzyme to transcribe the genetic information in the newly introduced DNA to generate the sgRNA. Another method for making sgRNA, termed in vitro transcription (IVT), involves transcribing the sgRNA from the corresponding DNA sequence outside the cell. First, a DNA template is designed that contains the guide sequence and an additional RNA polymerase promoter site upstream of the sgRNA sequence, The sgRNA is then transcribed using kits that contain reagents and recombinant RNA polymerase. Kits are commercially available, for example, TranscriptAid T7 High Yield Transcription Kit (ThermoFisher Scientic). Exemplary primers are provided in Table 1. An exemplary sgRNA used for the detection of SARS-Co-V-2 is SEQ ID NO:24.

### B. Lateral Flow Device

In preferred embodiments, the disclosed point-of-care assay is a lateral flow assay, in which the test sample flows along a solid substrate via capillary action. As shown in FIG. 6 a lateral flow device 10 includes a solid substrate 12, such as a membrane strip, having an application point 14, an optional conjugate zone 16, a capture zone 18, and an absorbent zone 20 (e.g., a wicking pad). Binding agents are optionally present in the conjugate zone 16. Capture agents are immobilized in the capture zone 18, which preferably one or more capture lines 22 for detecting captured analyte (s) (capture complex).

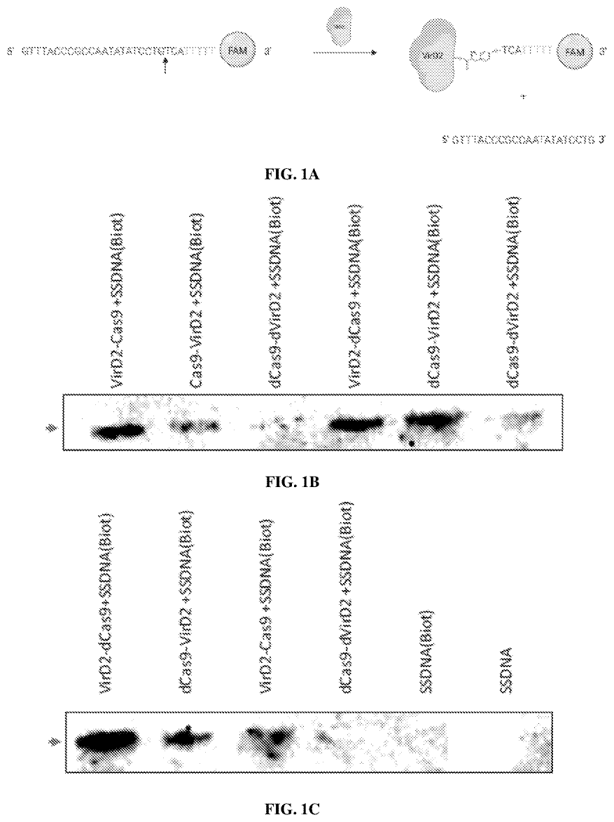

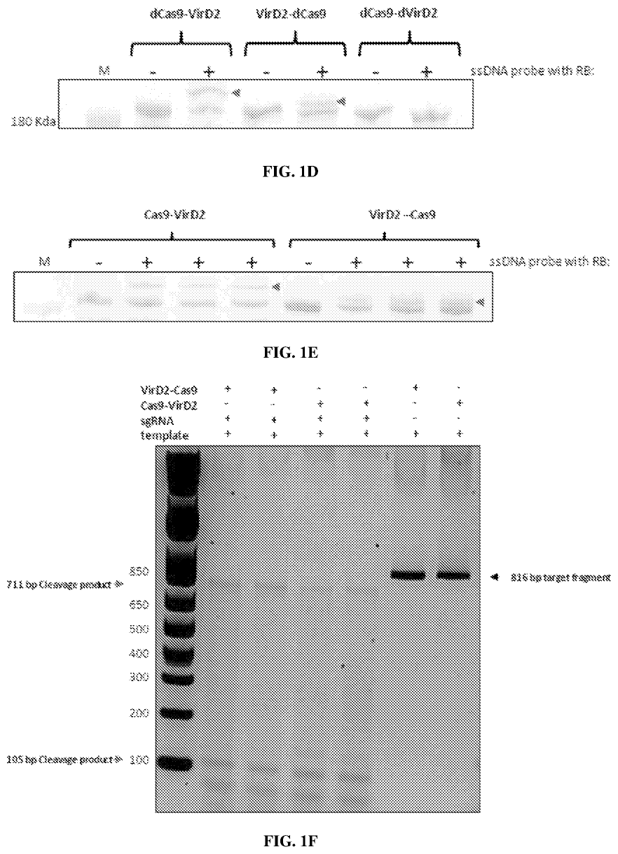

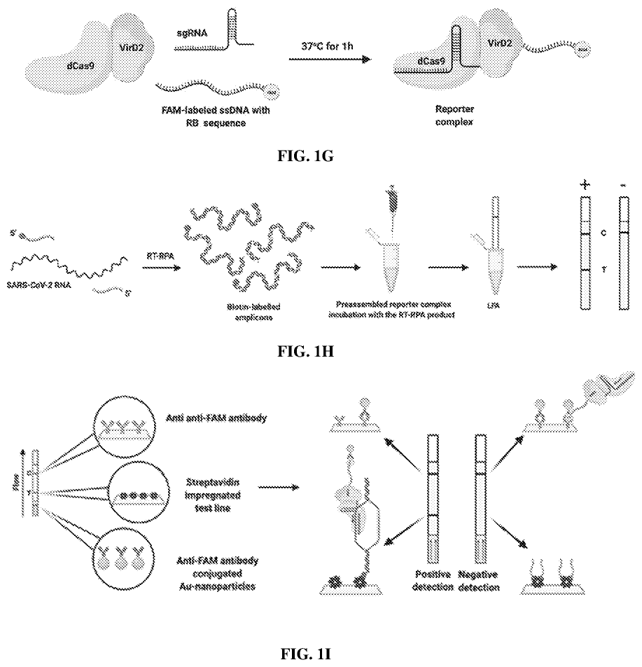

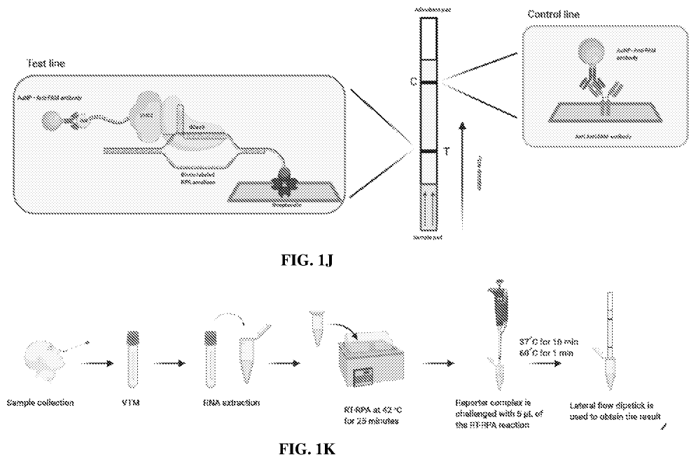

In a preferred embodiment, the capture zone includes a zone for detecting analyte in the sample and it includes binding partners to the label on the analyte and a second capture zone which includes binding partners for the second label on reporter molecule used in the assay. A simple lateral flow device is exemplified in FIG. 1H. The streptavidin-coated line (T) captures biotin-labelled amplicons (analyte of interest). The test line can include any member of an affinity pair used as affinity tags, in which case, its binding partner is used to label the target nucleic acid. Affinity tags are any molecular species which form highly specific, noncovalent, physiochemical interactions with defined binding partners. Affinity tags which form highly specific, noncovalent, physiochemical interactions with one another are defined herein as “complementary”. Suitable affinity tag pairs are well known in the art and include epitope/antibody, biotin/avidin, biotin/streptavidin, biotin/neutravidin, glutathione-S-transferase/glutathione, maltose binding protein/amylase and maltose binding protein/maltose. Examples of suitable epitopes which may be used for epitope/antibody binding pairs include, but are not limited to, HA, FLAG, c-Myc, glutatione-S-transferase, His6, GFP, DIG, biotin and avidin. Antibodies (both monoclonal and polyclonal and antigen-binding fragments thereof) which bind to these epitopes are well known in the art.

Affinity tags that are conjugated to adaptor elements allow for highly flexible, modular assembly and disassembly of functional elements which are conjugated to affinity tags which form highly specific, noncovalent, physiochemical interactions with complementary affinity tags which are conjugated to adaptor elements. Adaptor elements may be conjugated with a single species of affinity tag or with any combination of affinity tag species in any ratio. The ability to vary the number of species of affinity tags and their ratios conjugated to adaptor elements allows for exquisite control over the number of functional elements which may be attached to the nanoparticles and their ratios.

In another embodiment adaptor elements are coupled directly to functional elements in the absence of affinity tags, such as through direct covalent interactions. Adaptor elements can be covalently coupled to at least one species of functional element. Adaptor elements can be covalently coupled to a single species of functional element or with any combination of species of functional elements in any ratio.

In a preferred embodiment adaptor elements are conjugated to at least one affinity tag that provides for assembly and disassembly of modular functional elements which are conjugated to complementary affinity tags. In a more preferred embodiment, adaptor elements are fatty acids that are conjugated with at least one affinity tag. In a particularly preferred embodiment, the adaptor elements are fatty acids conjugated with avidin or streptavidin. Such avidin/streptavidin-conjugated fatty acids allow for the attachment of a wide variety of biotin-conjugated functional elements.

The adaptor elements are provided on, or in the surface of, nanoparticles at a high density. This high density of adaptor elements allows for coupling of the nanoparticle to a variety of species of functional elements while still allowing for the functional elements to be present in high enough numbers to be efficacious.

In a sample containing SARS-CoV-2, the reporter complex bound to the target DNA will accumulate gold (Au) nanoparticle-labeled anti-FAM, resulting in the visual detection at the test line. Gold nanoparticles and latex beads are the most widely used labels in commercial lateral flow immunoassays. The control line (C) is impregnated with anti-anti FAM antibodies that also accumulate Au nanoparticle-labeled anti-FAM and thus serve as a positive control. Commercially available lateral flow devices can be used. An exemplary lateral flow device is the device in the Universal HybriDetect-Universal Lateral Flow Assay Kit ( ) available from various compnies, for example, BioTrend CliniSciences Group; Cat# MGHD1; Milenia Biotech (cat number MGHD1).

The solid substrate 12, such as a membrane strip, can be made of a substance of sufficient porosity to allow movement of antibodies and analyte by capillary action along its surface and through its interior. Examples of suitable membrane substances include: cellulose, cellulose nitrate, cellulose acetate, glass fiber, nylon, polyelectrolyte ion exchange membrane, acrylic copolymer/nylon, and polyethersulfone. In a one embodiment, the membrane strip is made of cellulose nitrate (e.g., a cellulose nitrate membrane with a Mylar backing) or of glass fiber. In a preferred embodiment, the membrane strip is FUSION 5™ material (Whatman), which is a single layer matrix material that performs all of the functions of a lateral flow strip. For FUSION 5™, the optimal bead size is approximately 2 microns; the FUSION 5™ material has a 98% retention efficiency for beads of approximately 2.5 microns. Beads of 2.5 microns will not generally enter the matrix, whereas beads of below 1.5 microns will be washed out of the matrix.

The solid substrate 12 includes an application point 14, which can optionally include an application pad. For example, if the sample containing the analyte contains particles or components that should preferentially be excluded from the assay, an application pad can be used.

The solid substrate 12 includes an application point 14, which can optionally include an application pad. For example, if the sample containing the analyte contains particles or components that should preferentially be excluded from the immunoassay, an application pad can be used. The application pad typically can filter out particles or components that are larger (e.g., greater than approximately 2 to 5 microns) than the particles used in the disclosed methods.

The application pad may be used to modify the biological sample, e.g., adjust pH, filtering out solid components, separate whole blood constituents, and adsorb out unwanted antibodies. If an application pad is used, it rests on the membrane, immediately adjacent to or covering the application point. The application pad can be made of an absorbent substance which can deliver a fluid sample, when applied to the pad, to the application point on the membrane. Representative substances include cellulose, cellulose nitrate, cellulose acetate, nylon, polyelectrolyte ion exchange membrane, acrylic copolymer/nylon, polyethersulfone, or glass fibers. In one embodiment, the pad is a Hemasep™-V pad (Pall Corporation). In another embodiment, the pad is a Pall™ 133, Pall™ A/D, or glass fiber pad.

The solid substrate 12 optionally contains a conjugate zone 16, which contains binding agents. In some embodiments, the conjugate zone contains binding agents which bind the analyte to be measured and a control analyte. When the sample migrates through the conjugate zone containing binding agents, the analytes in the sample interacts with the binding agents to form capture complexes.

The absorbent zone 20 preferably contains a wicking pad. If a wicking pad is present, it can similarly be made from such absorbent substances as are described for an application pad. In a preferred embodiment, a wicking pad allows continuation of the flow of liquid by capillary action past the capture zones and facilitates the movement of non-bound agents away from the capture zones.

The capture zone 18 contains capture agent immobilized (e.g., coated on and/or permeated through the membrane) to the membrane strip. In preferred embodiments, the capture agent is conjugated to a capture particle that is immobilized in the capture zone

The capture zone 18 is preferably organized into one or more capture lines containing capture agents. In preferred embodiments, the capture zone contains a plurality of capture lines for multiplex analysis, i.e., detection of two or more analytes. In addition, the capture zone 18 may contain one or more control capture lines for detecting the presence of control analyte (i.e., control or calibration capture zone). In preferred embodiments the control analyte is a compound that is not normally present in any prescription or non-prescription drug, food, beverage, or supplement. Preferably, the control analyte capture reagent specifically binds the control analyte but does not interact with the sample analyte being measured.

### II. METHODS OF USING

Vigilant (VirD2-dCas9 guided and LFA-coupled nucleic acid test),) is coupled to reverse transcription-recombinase polymerase amplification is to detect pathogenic nucleic acid in a sample. To overcome these drawbacks with prior methods discussed herein, the Vigilant detection system couples the functions of the CRISPR-Cas9 and DNA relaxases for a robust LFA for virus pathogen nucleic acid detection that can be used for POC applications.

Cas9 helicase activity eliminates the need for the DNA denaturation step, which is required in conventional hybridization-based LFA. Vigilant provides critical features including short running time, compatibility with quick extraction protocols, and isothermal amplification, which make it a practical method to detect viruses and pathogens. The low cost, estimated at $10/reaction, makes it affordable and deployable in low-resource settings for large-scale screening of COVID-19 cases.

The Vigilant system is very sensitive and specific. In the Vigilant modality, specificity is ensured by a two-step process i.e. specific amplification and specific detection by complementarity of sgRNA (single guide RNA), thereby reducing the chance for a false positive signal. Clinical studies suggest that the risk of SARS-CoV-2 transmission decrease dramatically when the number of viruses drops below 1000 particles. /μL [34-36]. A recently developed model of mass pandemic surveillance suggests that assays with fast turnaround time able to detect 100 copies/μL would be adequate for efficient high-throughput screening [37]. The low LoD offered by Vigilant surpasses this limit and is comparable to conventional PCR-based methods and newer CRISPR-based approaches.

FIG. 1K outlines an embodiment of the method disclosed herein.

### Sample Collection

The method includes collecting one or more samples from a subject and/or extracting and purifying RNA from the samples. The sample is preferably a saliva or nasopharyngeal swab sample. The sample is preferably collected, stored and transported in a suitable medium. For example, viral samples are transported in VTM (viral transport media). Viral transport media is a solution that allows for the safe transfer of viral samples to a laboratory for analysis. In many cases, this is a liquid that creates a balanced buffer solution for maintaining a neutral pH, antimicrobial agents, a source of protein, and sucrose that serves as a preservative. Viral transport media is included in a sterile tube typically made of premium medical grade plastic. Some tubes may be formulated to contain buffered proteins and antibiotics that suppress the growth of potentially contaminating bacteria and fungi. A securely closed transport tube is critical for protecting the sample specimen from contamination during transport. Many tubes feature screw caps designed for secure closure and ease of use, while other transport tubes may feature a plug top for a secure seal. Some viral transport media and transport systems are formulated to work with a specific type of swab. Not all swab materials are suitable for viral testing. For example, nasopharyngeal swabs cannot be made with cotton as organic materials interfere with the polymerase chain reaction (PCR) testing process. Some manufacturers sell swab kits that include viral transport systems, to eliminate any confusion around the right components. Viral transport media are commercially available. Universal transport media (available from Puritan; SKU#: UT-100) may be used to transfer samples to laboratories for use in viral antigen detection test, PCR and rapid tests. These tubes may contain antimicrobial agents to minimize bacterial and fungal contamination, as well as glass beads that assist in releasing and dispersing the sample into the medium during laboratory vortexing. UTM®: Viral Transport is a Universal Transport Medium for Collection, Transport, and Preservation of Clinical Specimens for Viral Molecular Diagnostic Testing, including SARS-Co-V-2, chlamydia, mycoplasma or ureaplasma organisms. The transport medium comes in a plastic, screw cap tube and maintains organism viability for 48 hours at room or refrigerated temperature.

### Sample Nucleic Acid Amplification

The second stem includes extracting RNA from the collected samples using known RNA extraction protocols, followed by amplification of the nucleic acid in the sample. Typically, the purified RNAs are converted to DNA by a reverse transcription reaction using reverse transcriptase (Reverse transcription). At this stage, if the subject was infected with viral RNA for example, the complementary DNA (cDNA) fragments derived from the RNA viruses are generated. Then, the virus-originated DNA fragments are sufficiently amplified by the qPCR reaction to a detectable level (e.g., by a fluorescent signals). The one-step RT-qPCR platform can simultaneously achieve both the RT and qPCR reactions in a single tube. In one embodiment, the RNA is amplified using PCR. In another embodiment, nucleic acid in the sample is amplified by RT-RPA. In one embodiment, the target DNA is amplified using biotin-labeled primers. Examples are provided in Table 1.

### Reporter Complex Assembly

The Reporter Complex includes the chimeric fusion protein of a CRISPR cas enzyme, preferably a dead Cas enzyme and a relaxase, sgRNA specific for the nucleic acid of interest, and the ssDNA probe recognized by the relaxase enzyme.

The reporter complex is prepared by combining the Cas-relaxase fusion protein with sgRNA (designed to target gene of interest), and reporter-labelled relaxase-specific nucleic acid in an appropriate buffer, for the example, the RPA reaction buffer (50 mM Tris-HCl pH 8.0, 500 mM KCl, 250 mM NaCl, 5 mM DTT, 50 mM MgCl₂, 250 mM L-arginine, and 250 mM L-glutamic acid) or PCR reaction buffer (100 mM HEPES pH 8.0, 500 mM KCl, 250 mM NaCl, 5 mM DTT, 25 mM MgCl₂), and an effective amount of an ribonuclease inhibitor. The reaction is incubated preferably at about 37° C. for an effective amount of time to form the reporter complex.

### Lateral Flow Assay and Detection

Detection of the positive samples is achieved by running the lateral flow after amplification of the acid of interest, and incubation with the reporter complex. The amplification product of the nucleic acid of interest is mixed and incubated with the preassembled reporter, preferably at 37° C. The mixture is contacted with running buffer, preferably HybriDetect Assay buffer containing about 10% BSA and mixed. The running buffer preferably includes, 10-20 mM Tris HCL, 50-150 mM KCL, 25-75 mM Nacl, 1-10 mM MagCl₂, 0.1-10 mM DTT, 10-100 mM arginine, 10-100 mM glutamate at pH 8.0. An exemplary preferred buffer is 10 mM Tris-HCl; 100 mM KCl; 50 mM NaCl; 1 mM DTT; 10 mM MgCl₂; 50 mM arginine; 50 mM glutamate; pH=8.0 at 25° C. A lateral flow device is then inserted into the reaction mixture as soon as its control band appears. A positive reaction is indicated by the presence of the lower (test) band while the upper band represents the control ban (FIG. 1I).

Vigilant reaction takes around 35 minutes with preassembled reporter complex, which makes it ideal both for in-field and POC applications.

Most of the reagents used in this assay are readily available commercially or, as a VirD2-dCas9 fusion protein, can be easily produced in-house at a massive scale. Once assembled, the reporter complex shows remarkable stability in an aqueous solution, which facilitates its use in a high-throughput manner and alleviates the need to prepare fresh reagents before each detection assay. Additionally, reagents can be lyophilized, which secures the reagent stability and shelf life at room temperature, further simplifying the protocol and making the method fully compatible with POC requirements. A wide variety of molecular labels could be covalently attached to Cas9 via VirD2 in a way that does not necessitate expensive, laborious and complicated chemical methods.

Specific and preferred embodiments of the disclosed compositions and methods are exemplified below.

### EXAMPLES

### Materials and Methods

### Nucleic Acid Preparation

a) Plasmids, ssDNA Probe, and Oligos

VirD2-Cas9 and Cas9-VirD2 clones were used to purify the fusion proteins. Mutant VirD2-dCas9 (dead Cas9-VirD2), dCas9-VirD2 (VirD2-dead Cas9), and dCas9-dVirD2 (dead Cas9-dead VirD2) clones for expression of the respective fusion proteins were custom synthesized by GenScript. Guide RNAs for Cas9 experiments were designed using SnapGene and ordered as gBlocks from Integrated DNA Technologies under the T7 promoter for in vitro transcription. The FAM-labeled ssDNA probe and biotin-labeled oligos were ordered from Integrated DNA Technologies. Sequences of the plasmids and oligos are listed in the Table 1.

TABLE 1
Primer Sequences

| Name | Sequence | Experimental description |
|---|---|---|
| virD2 reporter- | 5′- | Short, FAM-labeled probe |
| FAM-S | /gtttacccgccaatatatcctgtcaTTTTT/ | for VirD2 |
| | 56-FAM/-3′ (SEQ ID NO: 1) | |
| virD2 reporter- | 5′- | Long, FAM-labeled probe |
| FAM-L | /gtttacccgccaatatatcctgtcaTTTTTT | for VirD2 |
| | TTTT/56-FAM/-3′ (SEQ ID NO: 2) | |
| virD2 reporter-B-L | 5′- | Long, biotin-labeled probe |
| | /gtttacccgccaatatatcctgtcaTTTT | for VirD2 used for |
| | TTTTTT/biotin/-3′ | Westernblot |
| | (SEQ ID NO: 3) | analysis of VirD2 nuclease- |
| | | and binding activities |
| PCR-F1-N3 | 5′/biotin/CCGAAGAGCTACCAG | Forward PCR primer used |
| | ACGAATTC/3′ (SEQ ID NO: 4) | for proof-of-concept assay |
| PCR-R2-N3 | 5′/ | Reverse PCR primer used |
| | TGTAGCACGATTGCAGCATTG/3′ | for proof-of-concept assay |
| | (SEQ ID NO: 5) | |
| gBlock-N3-sgRNA- | 5′/GTCTCAGGCATAATACGAC | gBlock template used for |
| 1 | TCACTATAGGccagaagctggacttcc | IVT of sgRNA targeting |
| | ctaGTTTTAGAGCTAGAAATAG | both the |
| | CAAGTTAAAATAAGGCTAGT | PCR and RT-RPA |
| | CCGTTATCAACTTGAAAAAG | generated amplicons |
| | TGGCACCGAGTCGGTGC/3′ | |
| | (SEQ ID NO: 6) | |
| RPA-STOPCovid-F- | 5′/biotin/actaagaaatctgctgctgaggctt | Used for primer screening |
| 1-B | ctaag (SEQ ID NO: 7) | |
| RPA-STOPCovid-R- | 5′/tgcgtcaatatgcttattcagcaaaatgac | Used for primer screening |
| 1 | (SEQ ID NO: 8) | |
| RPA-STOPCovid-F- | 5′/biotin/gtactgccactaaagcatacaatgt | Used for primer screening |
| 2-B | aacac (SEQ ID NO: 9) | |
| RPA-STOPCovid-R-2 | 5′/gttttgtatgcgtcaatatgcttattcagc | Used for primer screening |
| | (SEQ ID NO: 10) | |
| RPA-N3-F-1-B | 5′/biotin/agctaccagacgaattcgtggtggt | Selected RT-RPA fwd |
| | gacgg (SEQ ID NO: 11) | primer |
| RPA-N3-R-1 | 5′/ttgtagcacgattgcagcattgttagcagg | Used for primer screening |
| | (SEQ ID NO: 12) | |
| RPA-N3-F-2-B | 5′/biotin/aattggctactaccgaagagctacc | Used for primer screening |
| | agacg (SEQ ID NO: 13) | |
| RPA-N3-R-2 | 5′/acgattgcagcattgttagcaggattgcgg | Selected RT-RPA rev |
| | (SEQ ID NO: 14) | primer |
| pstv-cnt-RB-ssDNA | cagatcaaTTCTCTTAGGTTTACC | Covalent binding |
| | CGCCAATATATCCTGTCAAA | confirmation of |
| | CACTGATAGTTTtcaca | VirD2.dCas9 fusions |
| | gtagGGTG (SEQ ID NO: 15) | |

b) In Vitro Transcription of sgRNA

In vitro transcription was performed using TranscriptAid T7 High Yield Transcription Kit (Thermo Scientific) following the manufacturer's instructions. Briefly, 10 μL of 5× TranscriptAid Reaction Buffer, 20 μL NTP mix, 10 μL of the DNA template (annealed sgRNA gBlock and T7 promoter oligo), 0.5 μL of RNase Out, 5 μL of TranscriptAid Enzyme Mix, and 4.5 μL of DEPC-treated water were incubated at 37° C. for 8 hours. In vitro transcribed RNA was purified using Direct-zol RNA MiniPrep Kit (Zymo Research). Production of the proper size sgRNA fragments was confirmed on a 2% agarose gel run in Tris-Borate-EDTA buffer.

### Protein Purification

Protein purification was performed as previously described [13]. Briefly, a single colony of BL21(DE3) was grown in 2X-YT media and induced at 0.6 OD₆₀₀ with 0.3 mM IPTG and incubated at 18° C. for 15 h at 180 rpm. Proteins were isolated using affinity purification column and further purified by size fractionation using the AKTA pure system (Cytiva).

### Functional Characterization of In-House Produced Enzymes

### a) Cas9 Nuclease Activity Assay of the Fusion Proteins

Target fragment (SARS-CoV-2, N-gene fragment) was amplified by PCR for Cas9-based cleavage assays. Ribo-nucleoprotein particles of VirD2-Cas9, Cas9-VirD2, and catalytically dead mutants VirD2-dCas9, dCas9-VirD2 and dCas9-dVirD2 were assembled at 37° C. for 10 minutes in 16.3 μL reaction consisting of 250 nM of the respective protein, 250 nM sgRNA in the cleavage buffer (10 mM Tris-HCl pH 8.0, 50 mM NaCl, 10 mM MgCl₂). Following the incubation, 3.7 μL containing 150 ng of the target was added into the tube and the reaction was incubated for 1 hour at 37° C. The protein was denatured at 95° C. for 5 minutes, the reaction was cooled on ice for 3 minutes, and the DNA products were separated on a 2% agarose gel.

### b) VirD2 and Probe Covalent Binding Assay of the Fusion Proteins

Biotin-labeled probe (ssDNA harboring the T-DNA right border sequence) was mixed with the fusion proteins (VirD2-Cas9, Cas9-VirD2, and catalytically dead mutants VirD2-dCas9, dCas9-VirD2 and dCas9-dVirD2) at the ratio 1:1 (250 nM each) V2 buffer (10 mM Tris-HCl pH 8.0, 50 mM NaCl, 10 mM MgCl₂). The reaction mix was incubated at 37° C. for 60 minutes. Protein loading dye was added to the reaction and heated for 95° C. for 3 minutes. The complex was resolved on 10% NuPAGE (Invitrogen) for 3 h at 4° C. and transferred to nitrocellulose membrane for 3 h at 4° C. The membrane was immuno-blotted with anti-biotin mouse primary antibody 1:1000 (Cat. No. SC-53179, Santa Cruz) and anti-mouse secondary antibody 1:2000 (Cat. No. A3688, Sigma) and detected with chemiluminescent ECL solution (BioRad). Alternatively, for direct gel mobility shift, probe (64 bp ssDNA harboring the T-DNA right border sequence) was mixed with the fusion proteins (VirD2-Cas9, Cas9-VirD2, and catalytically dead mutants VirD2-dCas9, dCas9-VirD2 and dCas9-dVirD2) at the ratio 1:1 (250 nM each) V2 buffer (10 mM Tris-HCl pH=8.0, 50 mM NaCl, 10 mM MgCl₂). The reaction mix was incubated at 37° C. for 60 minutes. Protein loading dye was added to the reaction and heated for 95° C. for 3 minutes. The complex was resolved on 10% NuPAGE (Invitrogen) for 3 h at 4° C. Gel was stained with Coomassie brilliant blue stain (MBP). Gel photos were taken using Gel Doc XR (BioRad).

RT-RPA Reactions with Synthetic Targets

RT-RPA was performed using the TwistAmp Basic kit following the manufacturer's instructions. Briefly, a well-mixed 47.5-μL sample (1 μl RNA template, 2.4 μL of 10 μM biotin-labeled forward and 2.4 μL of unlabeled reverse primers, 29.5 μL of Rehydration buffer, 0.5 μL of SuperScript IV reverse transcriptase, 1 μL of RNase H, 0.5 μL of RNase Out, 10.2 μL H₂O) was added to the lyophilized RPA reaction components (TwistAmp Basic) and homogenized by pipetting. Magnesium acetate (2.5 μL of 280 mM) was added to each tube and mixed. The isothermal amplification was performed at 42° C. for 25 minutes. To confirm the DNA isothermal amplification, 10 μL of the reactions were purified using QIAquick PCR Purification Kit and separated on a 1.5 agarose gel.

### VirD2-dCas9 Reporter Complex Assembly

The reporter complex (final concentration of 250 nM each VirD2-dCas9, sgRNA and FAM-labeled reporter oligo) was prepared by combining 1.25 μL of 5 μM VirD2-Cas9 secondary stock, 1.25 μL of 5 μM sgRNA, 1.25 μL of 5 μM FAM-labeled reporter oligo, 5 μL of 5×RPA reaction buffer (50 mM Tris-HCl pH 8.0, 500 mM KCl, 250 mM NaCl, 5 mM DTT, 50 mM MgCl₂, 250 mM L-arginine, and 250 mM L-glutamic acid) or 5 μL 5X PCR reaction buffer (100 mM HEPES pH 8.0, 500 mM KCl, 250 mM NaCl, 5 mM DTT, 25 mM MgCl₂), 0.5 μL of RNase Out and ultra-pure water to 20 μL. The reaction was incubated at 37° C. for 60 minutes. Two reactions, 20 μL each, were combined in a single tube to make 40 μL preassembled reporter reaction.

### VirD2-dCas9 Detection Assay and Lateral Flow Assay

RT-RPA product (5 μL) was mixed with 40 μL of preassembled reporter. The 45-μL reaction mix was incubated at 37° C. for 10 minutes followed by 1 minute at 60° C. Following the incubation, 55 μL of the running buffer (44.5 μL of the HybriDetect Assay Buffer with 10.5 μL of 10% BSA) was added directly into the reaction and mixed. Room temperature adjusted HybriDetect Dipsticks were placed into the tube containing the reaction mixture. Lateral flow strips were removed from the tube as soon as the control band appeared, and the result was called within 10 minutes. Images of the strips were taken within 25 minutes after the beginning of the LFA.

### Limit of Detection Assay

SARS-CoV-2 RNA (synthetic RNA from IDT) was diluted to final concentrations corresponding to 1, 2.5, 7.5, 10, 50, and 100 copies/μL or 50, 125, 375, 500, 2500, and 5000 copies/reaction. The respective volume of RNA sample was added to the RT-RPA reactions. Nuclease-free water was used as the negative control. Following the RT-RPA reaction, 5 μL of the product was transferred into the reaction containing the preassembled reporter complex and the samples were detected as previously described. The detection limit was considered as the concentration that could be successfully detected within 10 minutes of the LFA assay in all three replicates.

Validation of the Developed Protocol with SARS-CoV-2 Clinical Sample

RNA samples from SARS-CoV-2 RT-PCR positive (26 clinical samples) and negative (4 clinical samples) were used for evaluation of the Vigilant protocol. RNA (4 μL) was added to the RT-RPA reaction and 5 μL of the amplified product was used for detection in the next step.

### Results

### Design and Construction of Vigilant for Nucleic Acid Detection

The studies herein were based on a hypothesis that a fusion of a Crisper enzyme exemplified herein using Cas9 or dCas9 (specifically, SpCas9 or SpdCas9) and a relaxase, exemplified herein using VirD2 could be exploited to develop a detection platform by harnessing the unique properties and characteristics of each protein. Since VirD2-Cas9 fusion protein can remain bound to specifically designed ssDNA sequences; this led to the hypothesis that a short oligonucleotide with FAM at its 3′-end could remain covalently attached to the Tyr29 after VirD2-dCas9 recognition. To demonstrate that VirD2 in the fusion protein can successfully bind a 3′-end-labeled ssDNA of interest (FIG. 1A), a ssDNA consisting of a specific 25-bp VirD2 recognition sequence and a 5-T nucleotide bridge was incubated with a biotin label at the 3′ end. Western blot analysis detected the presence of the biotin-labeled moiety attached to the protein, thus confirming the activity of VirD2 protein in the fusion construct (FIG. 1B-C). To confirm the ssDNA binding ability of the disclosed fusion modules, gel mobility shift assays were performed. Incubation of a 64-bp ssDNA (harboring the T-DNA right border sequence) with VirD2-Cas9 fusion proteins demonstrated a mobility shift of the VirD2-Cas9—DNA complex (FIG. 1D and 1E). The results demonstrated that the fusion modules in both orientations can bind ssDNA; however, a different shift pattern for Cas9-VirD2 was observed. This different shift might be due to steric interferences that arise due to the subunits' spatial orientation. Additional studies then demonstrated that the Cas9 part of the complex retains its enzymatic activity of binding and cleaving a target DNA (FIG. 1F). Therefore, each component of the fusion is fully active.

Subsequent studies were based on the hypothesis that the resulting complex could be used as a reporter to detect isothermally amplified, biotin-labeled amplicons. In the proposed module, Cas9 provides target-specific binding and VirD2 carries a 3′ FAM-labeled oligonucleotide probe for detection. To assemble the required components, FAM-labeled oligonucleotide were a design that contained a 25-nt VirD2 recognition sequence at the 5′ end and a short sequence containing five or ten thymines, followed by FAM at the 3′ end. VirD2 cleaves in the recognition sequence and remains covalently bound to the short oligonucleotide probe labeled with FAM at its 3′ end.

Next a sgRNA was designed to targetSARS-CoV-2 N gene. sgRNA complementarity will bring the VirD2-dCas9-ssDNA-FAM N gene sequence. As the final part of the complex, biotin-labeled primers were designed to amplify the target DNA, which was reverse-transcribed and amplified from the SARS-CoV-2 N gene via polymerase enzyme. When these components are assembled, the resulting structure (Biotin-DNA+VirD2-Cas9-sgRNA-ssDNA-FAM) is both biotin and FAM-labeled and can therefore be detected using commercially available lateral flow strips (FIG. 1G-I).

### Vigilant Specifically Detects Nucleic Acids

Next, a proof-of-principle detection assay was performed with amplicon targets generated by PCR, targeting the N gene of the SARS-CoV-2 genome. The reporter complex consisting of the VirD2-Cas9 fusion protein, sgRNA, and FAM-labeled ssDNA oligonucleotide were preassembled at 37° C. for 1 hour. Following amplification of the N gene, 5 μL of the unpurified, biotin-labeled PCR product was transferred to the reaction containing preassembled reporter complex and the mixture was incubated at 37° C. for 10 minutes to assemble the sgRNA-VirD2-Cas9-ssDNA-FAM complex and at 60° C. for 1 minute to release any un-specifically bounded DNA.

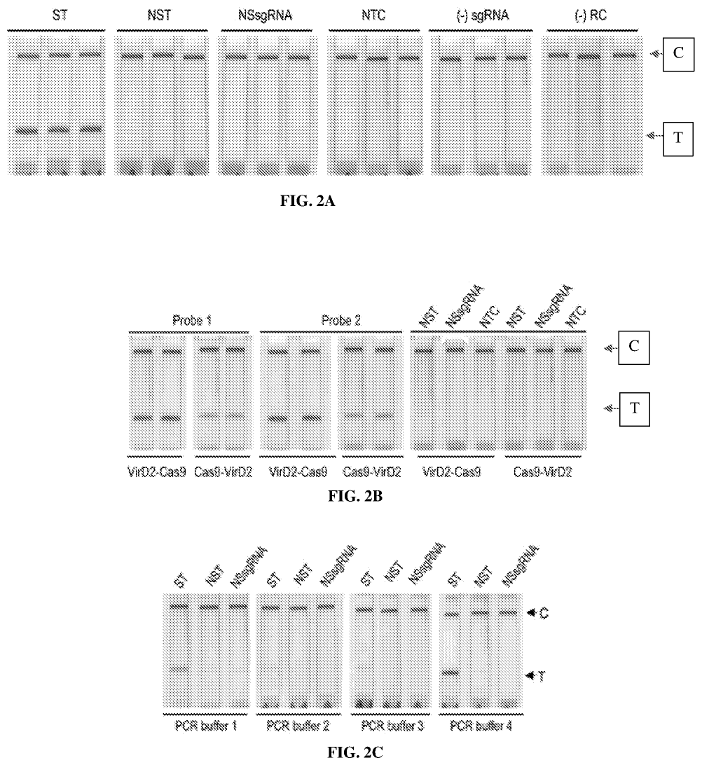

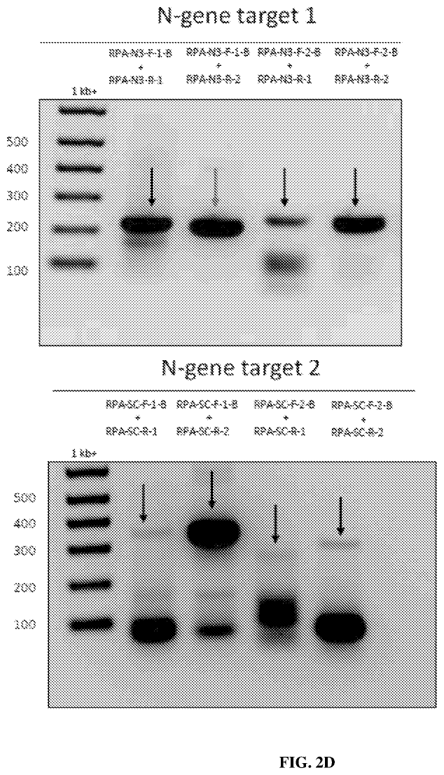

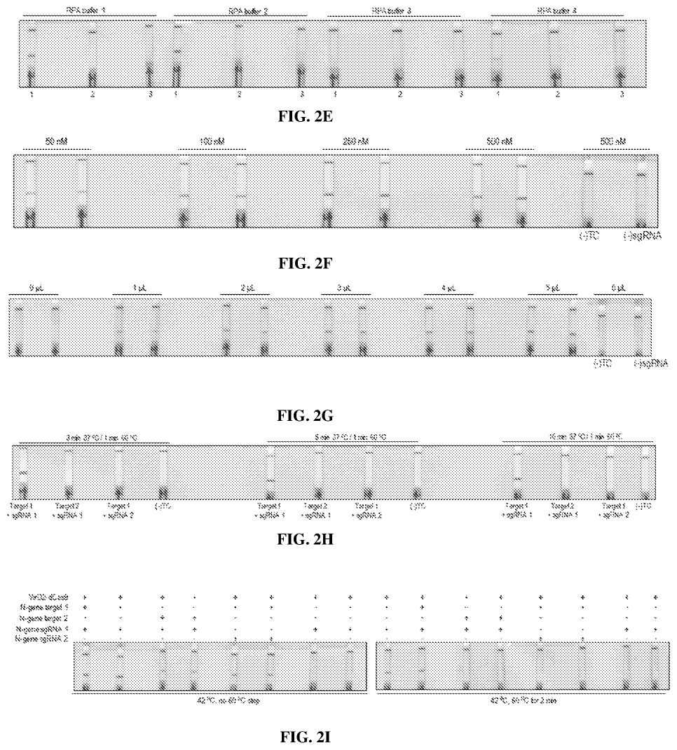

Following the addition of the running buffer, the reaction was applied to the lateral flow strip and the band at the test line appeared within 3 minutes specifically in the samples containing the correct amplicons (FIG. 2A). To test the parameters for the disclosed reporter system, the complex using VirD2 and Cas9 in both orientations (VirD2-Cas9 or Cas9-VirD2) was assembled and probes containing short (five-T) and long (ten-T) spacers were used. The experimental results indicate that VirD2-Cas9 with both short and long spacer probes display superior performance compared to Cas9-VirD2 (FIG. 2B).

Performance with PCR products as a function of buffer compositions were tested. PCR buffer 4 showed better detection than the other buffers used (FIG. 2C). This demonstrates that the buffer composition plays a major role in the performance of the assay. The reaction conditions for VirD2-Cas9 for specific and sensitive detection of nucleic acids were characterized. Buffer 1 (20 mM HEPES; 100 mM KCl; 50 mM NaCl; 1 mM DTT; 5 mM MgCl2 ; pH=8.0); Buffer 2 (20 mM HEPES; 100 mM KCl; 50 mM NaCl; 5 mM MgCl2; pH=8.0); Buffer 3 (20 mM Tris-HCl; 100 mM KCl; 50 mM NaCl; 5 mM MgCl2; 50 mM arginine; 50 mM glutamate; pH=8.0 at 25° C.); Buffer 4 (used in Viglant): 10 mM Tris-HCl; 100 mM KCl; 50 mM NaCl; 1 mM DTT; 10 mM MgCl2; 50 mM arginine; 50 mM glutamate; pH=8.0 at 25° C.

### Vigilant Demonstrates Robust Detection of RT-RPA Products

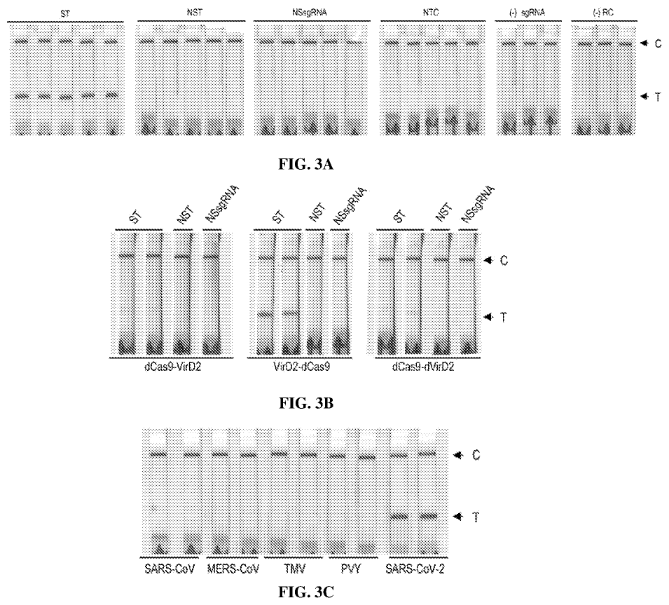

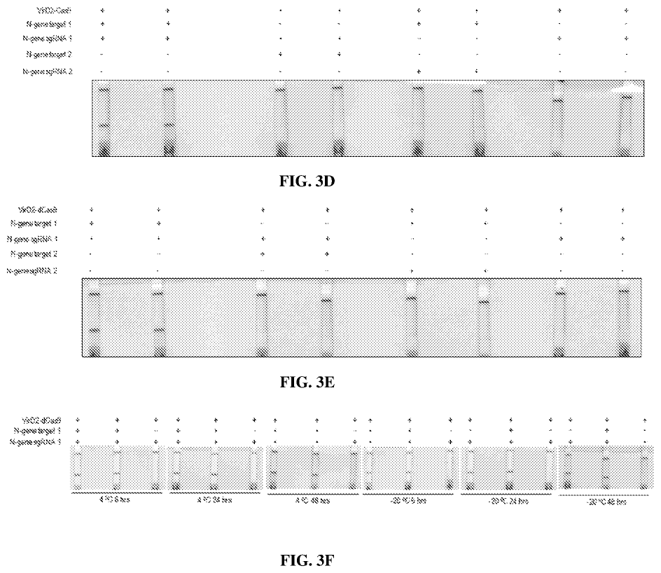

Due to its simplicity and rapidity, RPA has been widely used for the detection of SARS-CoV-2 coupled to LFAs via Cas12 and Cas13 CRISPR based detection methods. Subsequent studies therefore tested whether the Vigilant system is capable of detecting RT-RPA products (FIG. 1H). To this end, four sets of RPA primers were designed each targeting two different regions within the SARS-CoV-2 nucleocapsid gene (N) and screened them for optimal amplification performance (FIG. 2D). After selecting the best-performing set, 5 μL of the RPA amplification mixture was exposed to the preassembled reporter complex. The experimental results yielded a positive result at the test line on the lateral flow strips in the reactions containing only the correct amplicons while showing no bands at the test line in control reactions, including the nonspecific amplicon of SARS-CoV-2 N-gene (FIG. 3A). RT-RPA reactions were further optimized by screening for the most effective reaction buffer, reporter complex concentration, RT-RPA input volume and assay time (FIG. 2E-H). To confirm that Vigilant is compatible with the temperature required for most of the reverse transcriptases, RT-RPA was performed at 42° C. (FIG. 2I).

Inactivation of the Cas9 nuclease catalytic activity does not impair its DNA binding activity, as binding to the target depends on sgRNA complementarity rather than cleavage. Fusions of dCas9 with VirD2 were generated and deactivated VirD2 (dVirD2) and compared their activity with VirD2-Cas9 for target detection. Subsequently, the VirD2-dCas9 module was tested in the RT-RPA assays. The VirD2-dCas9 module demonstrated a robust detection of the SARS-CoV2 N gene RT-RPA product compared to the other two modules (FIG. 3B). The data show that VirD2-dCas9 is capable of specific nucleic acid detection.

Next, in order to demonstrate the specificity of Vigilant, MERS-CoV and SARS-CoV2 N gene template DNAs were used as controls. A plant RNA virus was also used as control. The Vigilant assay showed no cross-reactivity with the non-specific targets and specifically detected only SARS-CoV-2 RNA (FIG. 3C).

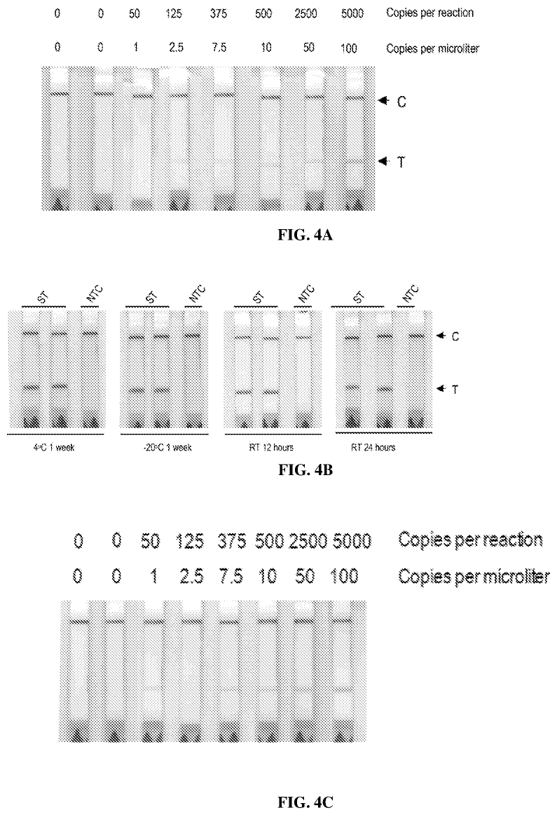

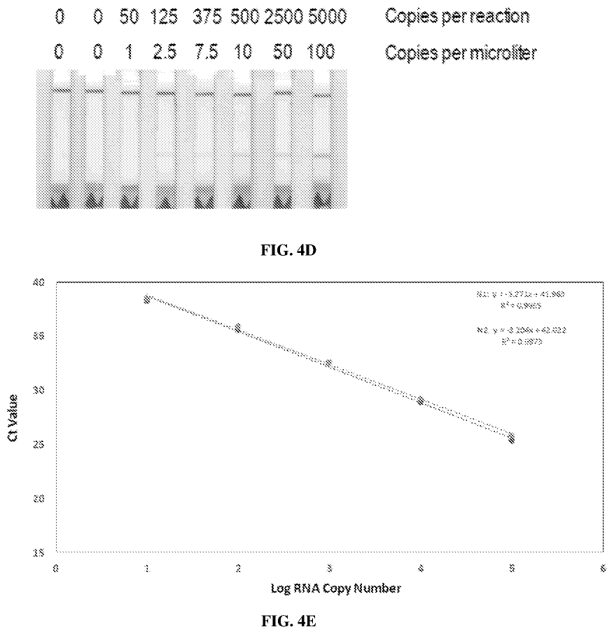

The Vigilant module possesses high sensitivity and stability Assay sensitivity is an essential characteristic for any detection platform. To determine the limit of detection (LoD) of the Vigilant system for nucleic acid detection, dilutions of 0, 1, 2.5, 7.5, 10, 50, 100 copies/μL of synthetic SARS-CoV-2 genomic RNA was tested in the RT-RPA reaction. Mixing of the RT-RPA amplified product of each dilution with the reporter complex determined the LoD as 2.5 copies/μL (FIG. 4A and 4C-4D). For comparison, the Ct values of the serial dilutions of the synthetic RNA were measured to determine the clinical relevance of the LoD for the detection of clinical samples by RT-qPCR (FIG. 4E). The assays showed that Vigilant could detect as little as 2.5 copies/μL of the synthetic RNA, which corresponds to 40-60 copies/reaction of virus, a concentration that is clinically relevant.

Subsequent studies evaluated the stability of the reporter complex to determine shelf-life and storage requirements. The reporter complex was preassembled and stored directly in the reaction buffer at room temperature for 6, 12, and 24 hours. Additionally, the preassembled reporter complex was stored at 4° C. and −20° C. for 24 h, 48 h, and one week. After performing the detection reactions with stored reagents, no decrease in performance was observed, indicating that the reagents can be stored at room temperature in an aqueous solution at 4° C. or −20° C. for at least one week (FIG. 3F and 4B).

### Vigilant Validation in COVID-19 Clinical Samples

Next, Vigilant was validated for the detection of SARS-CoV-2 in clinical samples. The detection of the signal in clinical samples by as a function of the input RNA concentration and the amount of the RT-RPA added to the Vigilant detection complex was investigated. To exclude any sample bias, SARS-CoV2 clinical samples with wide range of Ct values were used. To avoid experimental bias, the positive and negative samples were randomized, the Vigilant results recorded, and compared these to the RT-qPCR data. This validation was conducted using 26 positive samples and 4 negative samples based on RT-qPCR (table 2).

TABLE 2
Designation and the Ct value of the clinical samples used in this
study. RT-qPCR was performed on Trizol based isolated RNA.

| | Ct value | | Ct value |
|---|---|---|---|
| Sample | (RT- | Sample | (RT- |
| designation | qPCR) | designation | qPCR) |
| 155418 | 21 | 155960 | 15 |
| 155378 | 34 | 155974 | 26 |
| 155381 | 21 | 156852 | 21 |
| 155383 | 21 | 161058 | 17 |
| 155384 | 14 | 161063 | 16.5 |
| 155385 | 22 | S1504 | 30.75 |
| 155388 | 22 | S1611 | 31.12 |
| 155389 | 23 | S1605 | 31.45 |
| 155369 | 16 | S1547 | 31.52 |
| 155397 | 18 | S1606 | 31.52 |
| 155398 | 23 | S1560 | 31.57 |
| 155902 | 15 | NID-1 | >38 |
| 155905 | 20 | NID-2 | >38 |
| 155908 | 17 | NID-3 | >38 |
| 155916 | 22 | NID-4 | >38 |
| K0250 | 34.64 | S-573 | 33.25 |
| K0252 | 36.06 | S-270 | 33.80 |
| K0253 | 35.57 | S-272 | 32.70 |
| K0367 | 35.16 | S-263 | 33.29 |
| S-161 | 30.00 | S-766 | 31.99 |
| S-1541 | 30.55 | 161098 | 29 |
| S-1504 | 30.75 | 161100 | 20 |
| S-1611 | 30.77 | 161115 | 16 |
| S-1605 | 31.12 | 161117 | 15 |
| S-1547 | 31.45 | 161122 | 19 |
| S-1606 | 31.52 | 161123 | 22 |
| S-1560 | 31.57 | 161159 | 21 |
| S-437 | 32.47 | 161130 | 18 |
| S-578 | 33.25 | 161144 | 23 |
| S-586 | 32.88 | 61149 | 19 |
| K0250 | 34.64 | | |

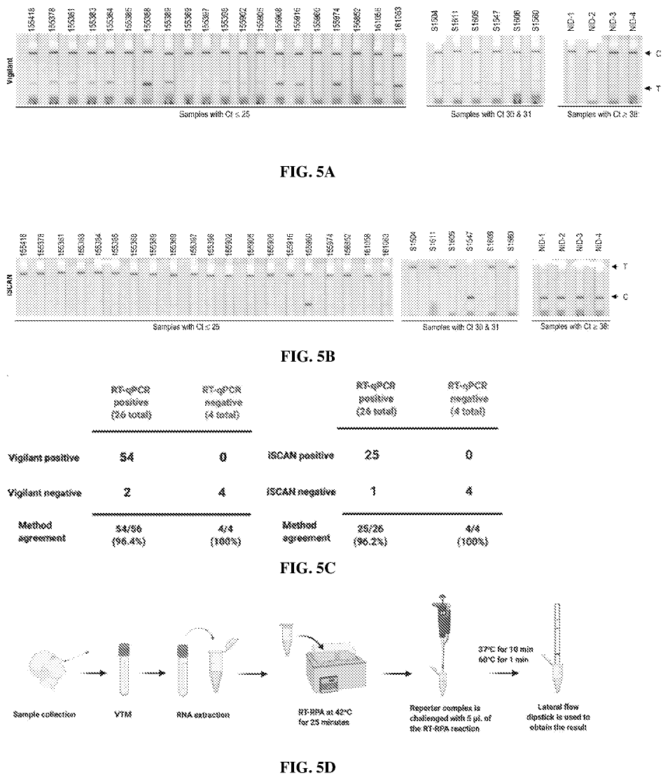

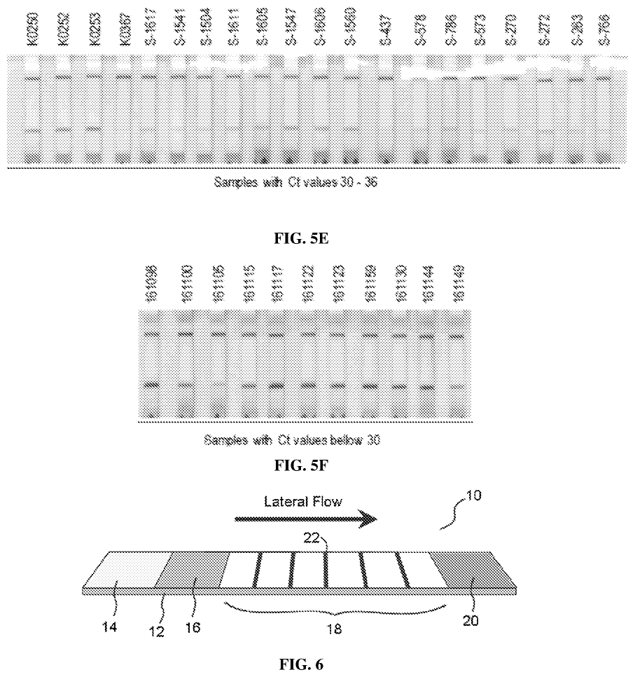

The data show that Vigilant exhibits high sensitivity, detecting 54 out of 56 positive samples. Moreover, 4 out of 4 negative samples were also negative by Vigilant. This indicates a 96.4% sensitivity and 100% specificity in agreement with RT-qPCR positive and negative samples, respectively (FIG. 5B).

Previous reports have indicated that the use of LFA for samples with Ct values >32 was not reproducible compared with fluorescent detection, where samples with Ct values up to 35/36 could be detected. The Vigilant system was compared with the CRISPR-Cas12-based iSCAN system in SARS-CoV-2 clinical samples. The data show that iSCAN (lateral flow readout) detected 25/26 positive samples and all 4/4 negatives were in agreement with RT-qPCR and Vigilant detected 54/56 positive 4/4 negatives in agreement with qPCR (FIGS. 5B and C). Therefore, the Vigilant LFA system exhibits good concordance, including sensitivity and specificity, with RT-qPCR and CRISPR-Cas12 based detection system and offers key features essential for effective POC testing (FIG. 5D).

### Discussion

Vigilant introduces a new class of CRISPR-based detection that provides critical features for powerful POC detection systems that do not rely on the trans, collateral, or cis activities of CRISPR enzymes [3, 38, 39]. The Vigilant principle can be applied not only to Cas9 variants but also to Cas12 and Cas14 variants, where chimeric fusions between a CRISPR enzyme and a relaxase can provide the dual functions of specific binding to the target sequence and the binding to a ssDNA probe resulting in a molecular complex for LFA detection. Moreover, PAM-independent Cas9 variants capable of the recognition of any DNA sequence in a PAM-independent manner can also be employed in the Vigilant system. This bypasses the need to find a PAM sequence for the binding site and expand the utility of this platform to any nucleic acid sequence. Similarly, other relaxases with different recognition sequences can be employed for binding to a ssDNA probe. Thus, generation of LFA based on the Vigilant principle can employ different CRISPRs and relaxases.

### REFERENCES

1. Kellner, M.J., et al., SHERLOCK: nucleic acid detection with CRISPR nucleases. Nature protocols, 2019. 14(10): p. 2986-3012.
2. Kilic, T., R. Weissleder, and H. Lee, Molecular and Immunological Diagnostic Tests of COVID- 19: Current Status and Challenges. iScience, 2020. 23(8): p. 101406.
3. Ackerman, C.M., et al., Massively multiplexed nucleic acid detection with Cas13. Nature, 2020. 582(7811): p. 277-282.
4. Puig, H.d., et al., Point-of-Care Devices to Detect Zika and Other Emerging Viruses. Annual Review of Biomedical Engineering, 2020. 22(1): p. 371-386.
5. Kosack, C.S., A.L. Page, and P.R. Klatser, A guide to aid the selection of diagnostic tests. Bull World Health Organ, 2017. 95(9): p. 639-645.
6. Wang, C., et al., A novel coronavirus outbreak of global health concern. Lancet, 2020. 395(10223): p. 470-473.
7. Sheridan, C., COVID-19 spurs wave of innovative diagnostics. Nat Biotechnol, 2020. 38(7): p. 769-772.
8. Jinek, M., et al., A programmable dual-RNA-guided DNA endonuclease in adaptive bacterial immunity. Science, 2012. 337(6096): p. 816-21.
9. Cong, L., et al., Multiplex genome engineering using CRISPR/Cas systems. Science, 2013. 339(6121): p. 819-23.
10. Piatek, A., et at, RNA-guided transcriptional regulation in planta via synthetic dCas9-based transcription factors. Plant Biotechnol J, 2015. 13(4): p. 578-89.
11. Ali, Z., et al., Efficient Virus-Mediated Genome Editing in Plants Using the CRISPR/Cas9 System. Mol Plant, 2015. 8(8): p. 1288-91.
12. Malina, A., et al.,Repurposing CRISPR/Cas9 for in situ functional assays. Genes Dev, 2013. 27(23): p. 2602-14.
13. Broughton, J.P., et al., CRISPR-CasI2-based detection of SARS-CoV-2. Nat Biotechnol, 2020. 38(7): p. 870-874.
14. Ali, Z., et al., iSCAN: An RT-LAMP-coupled CRISPR-Cas12 module for rapid, sensitive detection of SARS-CoV-2. Virus Res, 2020. 288: p. 198129.
15. Arizti-Sanz, J., et al., Streamlined inactivation, amplification, and Cas13-based detection of SARS-CoV-2. Nat Commun, 2020. 11(1): p. 5921.
16. Wang, X., et al., Clustered Regularly Interspaced Short Palindromic Repeats/Cas9-Mediated Lateral Flow Nucleic Acid Assay. ACS Nano, 2020. 14(2): p. 2497-2508.
17. Vogel, A.M. and A. Das, Mutational analysis of Agrobacterium tumefaciens virD2: tyrosine 29 is essential for endonuclease activity. J Bacteriol, 1992. 174(1): p. 303-8.
18. Pelczar, P., et al., Agrobacterium proteins VirD2 and VirE2 mediate precise integration of synthetic T-DNA complexes in mammalian cells. EMBO Rep, 2004. 5(6): p. 632-7.
19. Xiao, X., et al., Agrobacterium tumefaciens-mediated transformation: an efficient tool for targeted gene disruption in Talaromyces marneffei. World J Microbiol Biotechnol, 2017. 33(10): p. 183.
20. Piers, K.L., et al., Agrobacterium tumefaciens-mediated transformation of yeast. Proc Natl Acad Sci U S A, 1996. 93(4): p. 1613-8.
21. Rolloos, M., P.J. Hooykaas, and B.J. van der Zaal, Enhanced targeted integration mediated by translocated I-SceI during the Agrobacterium mediated transformation of yeast. Sci Rep, 2015. 5: p. 8345.
22. Ali, Z., et at, Fusion of the Cas9 endonuclease and the VirD2 relaxase facilitates homology-directed repair for precise genome engineering in rice. Commun Biol, 2020. 3(1): p. 44.
23. Jauset-Rubio, M., et al., Ultrasensitive, rapid and inexpensive detection of DNA using paper based lateral flow assay. Sci Rep, 2016. 6: p. 37732.
24. Sagcan, H. and N. Turgut Kara, Detection of Potato ring rot Pathogen Clavibacter michiganensis subsp. sepedonicus by Loop-mediated isothermal amplification (LAMP) assay. Sci Rep, 2019. 9(1): p. 20393.
25. Li, S., et al., Lateral flow biosensor combined with loop-mediated isothermal amplification for simple, rapid, sensitive, and reliable detection of Brucella spp. Infect Drug Resist, 2019. 12: p. 2343-2353.
26. Qian, J., et al., An enhanced isothermal amplification assay for viral detection. bioRxiv, 2020.
27. Fu, S., et al., Probe-free label system for rapid detection of Cronobacter genus in powdered infant formula. AMB Express, 2018. 8(1): p. 155.
28. Zhang, Q., et al., The post-PAM interaction of RNA-guided spCas9 with DNA dictates its target binding and dissociation. Sci Adv, 2019. 5(11): p. eaaw9807.
29. Fozouni, P., et al., Amplification-free detection of SARS-CoV-2 with CRISPR-Cas13a and mobile phone microscopy. Cell, 2021. 184(2): p. 323-333.e9.
30. Palaz, F., et al., CRISPR-based tools: Alternative methods for the diagnosis of COVID-19. Clin Biochem, 2021.
31. Azhar, M., et al., Rapid, field-deployable nucleobase detection and identification using FnCas9. bioRxiv, 2020: p. 2020.04.07.028167.
32. Ma, H., et al., CRISPR-Cas9 nuclear dynamics and target recognition in living cells. J Cell Biol, 2016. 214(5): p. 529-37.
33. Anders, C. and M. Jinek, In vitro enzymology of Cas9. Methods Enzymol, 2014. 546: p. 1-20.
34. La Scola, B., et al., Viral RNA load as determined by cell culture as a management tool for discharge of SARS-CoV-2 patients from infectious disease wards. Eur J Clin Microbiol Infect Dis, 2020. 39(6): p. 1059-1061.
35. Quicke, K., et al., Longitudinal Surveillance for SARS-CoV-2 RNA Among Asymptomatic Staff in Five Colorado Skilled Nursing Facilities: Epidemiologic, Virologic and Sequence Analysis. medRxiv, 2020.
36. Wölfel, R., et al., Virological assessment of hospitalized patients with COVID-2019. Nature, 2020. 581(7809): p. 465-469.
37. Larremore, D.B., et al., Test sensitivity is secondary to frequency and turnaround time for COVID-19 surveillance. medRxiv, 2020.
38. Chen, J.S., et al., CRISPR-Cas12a target binding unleashes indiscriminate single-stranded DNase activity. Science, 2018. 360(6387): p. 436-439.
39. Harrington, L.B., et al., Programmed DNA destruction by miniature CRISPR-Cas14 enzymes. Science, 2018. 362(6416): p. 839-842.

1. A method of detecting target nucleic acids in a sample comprising amplifying the target nucleic acid, contacting the amplified target nucleic acid with a chimeric fusion protein comprising a CRISPR cas enzyme-relaxase, represented by the general formula:
CAS-relaxase Formula I
or
relaxase-Cas Formula II,
wherein CAS is the Crispr-case enzyme;
an sgRNA specific for target nucleic acid, and a nucleic acid sequence specifically recognized by the relaxase (probe),
wherein the amplified target nucleic acid comprises a first label and the nucleic acid sequence specifically recognized by the relaxase comprises a second label.

2. The method of claim 1, wherein the CRISPR cas enzyme is selected from cas9, cas12 or cas13.

3. The method of claim 1, wherein the relaxes is selected from the group consisting of VirD2, TrwC relaxase and RepA relaxase.

4. The method of claim 1, wherein the relaxase is VirD2, and the nucleic acid sequence specifically recognized by the relaxase is a 25-bp VirD2 recognition sequence, wherein the spacer comprises 3-15 T nucleotides.

5. The method of claim 1, wherein: (a) the second label is FAM; (b) the first label comprises biotin; (c) wherein the fusion proteins has a formula represented by formula II and/or (d) the target nucleic acid is amplified by polymerase chain reaction or recombinase polymerase amplification.

6. (canceled)

7. (canceled)

8. (canceled)

9. The method of claim 1, comprising forming a reporter complex comprising the fusion protein, sgRNA and the probe, comprising contacting the fusion protein, sgRNA and probe at about 37° C. for an effective amount of time to form a reporter comprising the fusion protein, sgRNA and the probe.

10. The method of claim 9, comprising contacting the reporter complex with a sample comprising analyte of interest at about 37° C., followed by incubation at about 60° C.

11. The method of claim 1, comprising contacting the sample in running buffer with a lateral flow device, wherein the lateral device comprises binding partners to the first label in a first zone and bind partners to the second label in a second zone, wherein the running buffer comprises 10-20 mM Tris HCL, 50-150 mM KCL, 25-75 mM Nacl, 1-10 mM MagCl₂, 0.1-10 mM DTT, 10-100 mM arginine, 10-100 mM glutamate at pH 8.0.

12. The method of claim 11, wherein the running buffer further comprises BSA.

13. The method of claim 12, wherein the running buffer comprises 5-15% BSA, preferably, 10% BSA.

14. The method of claim 11, wherein the running buffer comprises bout 10 mM Tris HCL, about 100 mM KCL, about 50 mM NaCl, about 1 mM DTT, about 10 mM MgCl2, about 50 mM arginine and about 50 mM glutamate.

15. The method of claim 11, wherein the first zone comprises stretavisin and the second zone comprises anti anti-FAM antibodies, wherein a positive result is indicated by two visual lines and a negative result, by one line.

16. The method of claim 1, wherein the fusion protein is encoded by a nucleic acid sequence selected from the group consisting of SEQ ID NO:16, 17 and 18.

17. The method of claim 1, wherein: (a) the fusion protein is encoded by a nucleic acid sequence of SEQ ID NO:16; (b) the fusion protein is encoded by a nucleic acid sequence of SEQ ID NO:17; an/or (c) the fusion protein is encoded by a nucleic acid sequence of SEQ ID NO:18.

18. (canceled)

19. (canceled)

20. A Kit comprising compositions for detecting target nucleic acids in a sample, wherein the compositions comprise a chimeric fusion protein comprising a dead CRISPR cas enzyme (dCas) and a relaxase, and a nucleic acid to which the relaxase binds, wherein the dead CrisprCas enzyme has no nuclease activity.

21. The kit of claim 20, wherein the relaxase is VirD2 and the nucleic acid to which the relaxase binds comprises SEQ ID NO: 19.

22. The kit of claim 20 wherein the dCas is selected from dCas9, dcas12 or dcas13.

23. The kit of claim 20, wherein the dCas is dCas9.

24. The kit of claim 20, wherein the fusion protein is encoded by a nucleic acid sequence selected from the group consisting of SEQ ID NO:16, 17 and 18.

25. (canceled)

26. (canceled)

27. (canceled)


SEQUENCE LISTING (26 sequences)

SEQ ID NO: 1 - DNA, 30 bp, Artificial Sequence
  Synthetic VirD2 reporter
  misc_feature (30)..(30): FAM Label
gtttacccgccaatatatcctgtcattttt

SEQ ID NO: 2 - DNA, 35 bp, Artificial Sequence
  Synthetic virD2 reporter
  misc_feature (35)..(35): FAM Label
gtttacccgccaatatatcctgtcatttttttttt

SEQ ID NO: 3 - DNA, 35 bp, Artificial Sequence; identical to SEQ ID NO: 2
  Synthetic virD2 reporter
  misc_feature (35)..(35): Biotin Label

SEQ ID NO: 4 - DNA, 23 bp, Artificial Sequence
  Syntheic primer
  misc_feature (1)..(1): Biotin Label
ccgaagagctaccagacgaattc

SEQ ID NO: 5 - DNA, 21 bp, Artificial Sequence
  Synthetic primer
tgtagcacgattgcagcattg

SEQ ID NO: 6 - DNA, 124 bp, Artificial Sequence
  Synthetic gBlock-N3-sgRNA
gtctcaggcataatacgactcactataggccagaagctggacttccctagttttagagctagaaatagcaagttaaaataaggctagtccgttatcaacttgaaaaagtggcaccgagtcggtg

SEQ ID NO: 7 - DNA, 30 bp, Artificial Sequence
  Syntheti primer
  misc_feature (1)..(1): Biotin label
actaagaaatctgctgctgaggcttctaag

SEQ ID NO: 8 - DNA, 30 bp, Artificial Sequence
  Synthetic primer
tgcgtcaatatgcttattcagcaaaatgac

SEQ ID NO: 9 - DNA, 30 bp, Artificial Sequence
  Synthetic primer
  misc_feature (1)..(1): Biotin label
gtactgccactaaagcatacaatgtaacac

SEQ ID NO: 10 - DNA, 30 bp, Artificial Sequence
  Synthetic primer
gttttgtatgcgtcaatatgcttattcagc

SEQ ID NO: 11 - DNA, 30 bp, Artificial Sequence
  Synthetic primer
  misc_feature (1)..(1): Biotin Label
agctaccagacgaattcgtggtggtgacgg

SEQ ID NO: 12 - DNA, 30 bp, Artificial Sequence
  Synthetic primer
ttgtagcacgattgcagcattgttagcagg

SEQ ID NO: 13 - DNA, 30 bp, Artificial Sequence
  Synthetic primer
  misc_feature (1)..(1): Biotin Label
aattggctactaccgaagagctaccagacg

SEQ ID NO: 14 - DNA, 30 bp, Artificial Sequence
  Synthetic primer
acgattgcagcattgttagcaggattgcgg

SEQ ID NO: 15 - DNA, 69 bp, Artificial Sequence
  Synhthetic pstv-cnt-RB-ssDNA
cagatcaattctcttaggtttacccgccaatatatcctgtcaaacactgatagttttcacagtagggtg

SEQ ID NO: 16 - DNA, 10941 bp, Artificial Sequence
  Synthetic sequence encoding fusion protein
ccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccgaattcgagctccgtcgacaagcttgcggccgcatggacaagaagtacagcatcggcctggctatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggacgccatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaaagaaaaagagaaaagtatacccctacgacgtgcccgactacgcctgtggaggcggtggatctggcggtggcggaagtgggggaggtggaagtggtaccatgcccgatcgagctcaagttatcattcgcattgtgccgggaggtggcaccaagacccttcaacaaattatcaatcagttggaggccctatcccggaagggcaggctggagctgcagcgttcagcccgacatctcgatattcccctgccaccggatcaaatccacgaacttgcccgaagctgggttcaagagactggaacttatgacgaaagtcagccagacgaggaaaggcaacaggagttgaccacccatattattgtaagcttccccgccggtacaagccaggtagcggcttatgcggcgagccgggagtgggcagccgagatgtttgggtcaggcgcaggggggggccgatacaactatcttacggccttccacatcgatcgcgaccacccacatctgcatgtcgtcgtcaatcggcgcgaacttttaggacacggctggctgaagatatctcggcgccatccccaactgaattacgacgccctgcgcataaagatggccgagatttcacttcgtcatggcattgccctcgatgcgagccgacgagcagaacgtggcatcaccgagcggccgatcacttatgcccaatatcggcgccttgagcgggagcaggctcgccaaatccgtttcgaagacgcggatttggaacagtcgtcgccgcaaggagatcatccagaattcagccaacctttcgatacatccccatttgaagcatccgcgggcggaccggaggacatgcctcggcccaacaatcggcagaatgagtcgcaagttcatctccaggagccagctggtgtcagcaacgaagccggtgtccttgtgcgggttgcattggagacggagcgccttgctcaaccattcgtttccgaaaccattctcgcggacgacatagggagcggctcttcgcgtgttgccgagggccgtgtggagagcgcaaaccgcactcccgatattcctcgcgcagcaactgaagctgccacgcacacgacacacgaccggcagcggcgtgcaaagcgtcctcatgatgacgacggagggccgagtggagcaaaacgtgtgacattggaaggcatcgcggttgggccccaggcgaacgccggcgaacaggatggcagtagtggccccttagtacggcaagctggaacgtctcggccatctccaccgacggccacgacgcgggccagcaccgcaaccgattcattgtctgctacagcccacctccagcaacggagaggtgtcctttcaaagcgtccgcgtgaagatgatgatggagaaccgagtgaacgcaaacgcgagagagatgagcgcagcaaggacgggcgtgggggaaataggagataataagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagccaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattca

SEQ ID NO: 17 - DNA, 10938 bp, Artificial sequence
  Synhthetic sequence encoding fusion protein
tttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagccaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccgaattcgagctccgtcgacaagcttgcggccgcatgcccgatcgagctcaagttatcattcgcattgtgccgggaggtggcaccaagacccttcaacaaattatcaatcagttggagtatctatcccggaagggcaggctggagctgcagcgttcagcccgacatctcgatattcccctgccaccggatcaaatccacgaacttgcccgaagctgggttcaagagactggaacttatgacgaaagtcagccagacgaggaaaggcaacaggagttgaccacccatattattgtaagcttccccgccggtacaagccaggtagcggcttatgcggcgagccgggagtgggcagccgagatgtttgggtcaggcgcaggggggggccgatacaactatcttacggccttccacatcgatcgcgaccacccacatctgcatgtcgtcgtcaatcggcgcgaacttttaggacacggctggctgaagatatctcggcgccatccccaactgaattacgacgccctgcgcataaagatggccgagatttcacttcgtcatggcattgccctcgatgcgagccgacgagcagaacgtggcatcaccgagcggccgatcacttatgcccaatatcggcgccttgagcgggagcaggctcgccaaatccgtttcgaagacgcggatttggaacagtcgtcgccgcaaggagatcatccagaattcagccaacctttcgatacatccccatttgaagcatccgcgggcggaccggaggacatgcctcggcccaacaatcggcagaatgagtcgcaagttcatctccaggagccagctggtgtcagcaacgaagccggtgtccttgtgcgggttgcattggagacggagcgccttgctcaaccattcgtttccgaaaccattctcgcggacgacatagggagcggctcttcgcgtgttgccgagggccgtgtggagagcgcaaaccgcactcccgatattcctcgcgcagcaactgaagctgccacgcacacgacacacgaccggcagcggcgtgcaaagcgtcctcatgatgacgacggagggccgagtggagcaaaacgtgtgacattggaaggcatcgcggttgggccccaggcgaacgccggcgaacaggatggcagtagtggccccttagtacggcaagctggaacgtctcggccatctccaccgacggccacgacgcgggccagcaccgcaaccgattcattgtctgctacagcccacctccagcaacggagaggtgtcctttcaaagcgtccgcgtgaagatgatgatggagaaccgagtgaacgcaaacgcgagagagatgagcgcagcaaggacgggcgtgggggaaataggagaagatctggaggcggtggatctggcggtggcggaagtgggggaggtggaagtatggacaagaagtacagcatcggcctggctatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggacgccatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaaagaaaaagagaaaagtatacccctacgacgtgcccgactacgcctgttaagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaa

SEQ ID NO: 18 - DNA, 10941 bp, Artificial Sequence
  Synthetic sequence encloding fusion protein
ccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccgaattcgagctccgtcgacaagcttgcggccgcatggacaagaagtacagcatcggcctggctatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggacgccatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaaagaaaaagagaaaagtatacccctacgacgtgcccgactacgcctgtggaggcggtggatctggcggtggcggaagtgggggaggtggaagtggtaccatgcccgatcgagctcaagttatcattcgcattgtgccgggaggtggcaccaagacccttcaacaaattatcaatcagttggagtatctatcccggaagggcaggctggagctgcagcgttcagcccgacatctcgatattcccctgccaccggatcaaatccacgaacttgcccgaagctgggttcaagagactggaacttatgacgaaagtcagccagacgaggaaaggcaacaggagttgaccacccatattattgtaagcttccccgccggtacaagccaggtagcggcttatgcggcgagccgggagtgggcagccgagatgtttgggtcaggcgcaggggggggccgatacaactatcttacggccttccacatcgatcgcgaccacccacatctgcatgtcgtcgtcaatcggcgcgaacttttaggacacggctggctgaagatatctcggcgccatccccaactgaattacgacgccctgcgcataaagatggccgagatttcacttcgtcatggcattgccctcgatgcgagccgacgagcagaacgtggcatcaccgagcggccgatcacttatgcccaatatcggcgccttgagcgggagcaggctcgccaaatccgtttcgaagacgcggatttggaacagtcgtcgccgcaaggagatcatccagaattcagccaacctttcgatacatccccatttgaagcatccgcgggcggaccggaggacatgcctcggcccaacaatcggcagaatgagtcgcaagttcatctccaggagccagctggtgtcagcaacgaagccggtgtccttgtgcgggttgcattggagacggagcgccttgctcaaccattcgtttccgaaaccattctcgcggacgacatagggagcggctcttcgcgtgttgccgagggccgtgtggagagcgcaaaccgcactcccgatattcctcgcgcagcaactgaagctgccacgcacacgacacacgaccggcagcggcgtgcaaagcgtcctcatgatgacgacggagggccgagtggagcaaaacgtgtgacattggaaggcatcgcggttgggccccaggcgaacgccggcgaacaggatggcagtagtggccccttagtacggcaagctggaacgtctcggccatctccaccgacggccacgacgcgggccagcaccgcaaccgattcattgtctgctacagcccacctccagcaacggagaggtgtcctttcaaagcgtccgcgtgaagatgatgatggagaaccgagtgaacgcaaacgcgagagagatgagcgcagcaaggacgggcgtgggggaaataggagataataagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagccaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattca

SEQ ID NO: 19 - DNA, 25 bp, Artificial sequence
  Synthetic VirD2 recognition sequence
gtttacccgccaatatatcctgtca

SEQ ID NO: 20 - DNA, 5 bp, Artificial Sequence
  Synthetic nucleotide bridge
ttttt

SEQ ID NO: 21 - DNA, 15 bp, Artificial Sequence
  Synthetic nucleotiude spacer
ttttttttttttttt

SEQ ID NO: 22 - DNA, 10 bp, Artificial Sequence
  Synthetic nucleotide spacer
tttttttttt

SEQ ID NO: 23 - DNA, 96 bp, Artificial sequence
  Synthetic sgRNA
ccagaagctggacttccctagttttagagctagaaatagcaagttaaaataaggctagtccgttatcaacttgaaaaagtggcaccgagtcggtgc

SEQ ID NO: 24 - DNA, 29 bp, Artificial Sequence
  Synthetic sequence
tcgtattgtctatagcccagatttaagga

SEQ ID NO: 25 - DNA, 16 bp, Artificial Sequence
  Synthetic sequence
tgcgtattgtctatag

SEQ ID NO: 26 - DNA, 42 bp, Artificial Sequence
  Synthetic sequence
tgtgatagcgtgatttatcgcgctgcgttaggtgtatagcag